(19) United States
(12) Patent Application Publication
(10) Pub. No.: US 2023/0372403 A1
(43) Pub. Date: Nov. 23, 2023

(54) IMMUNOENGINEERING BIOMATERIALS FOR TREATMENT OF GRAFT REJECTION

(71) Applicant: THE REGENTS OF THE UNIVERSITY OF CALIFORNIA, OAKLAND, CA (US)

(72) Inventors: JONATHAN R.T. LAKEY, OAKLAND, CA (US); MOHAMMADREZA MOHAMMADI, OAKLAND, CA (US)

(21) Appl. No.: 18/248,213

(22) PCT Filed: Oct. 7, 2021
(86) PCT No.: PCT/US2021/053948

§ 371 Date: Apr. 6, 2023

Related U.S. Application Data
(60) Provisional application No. 63/089,726, filed on Oct. 9, 2020.

(51) Int. Cl. A61K 35/39; C12N 5/0775; C12N 5/00; A61K 9/50; A61K 39/00; A61P 3/10

(52) U.S. Cl. CPC A61K 35/39; C12N 5/0668; C12N 5/0012; A61K 9/5031; A61K 9/5068; A61K 9/5036; A61K 39/001; A61P 3/10; C12N 2533/74

(57) ABSTRACT

A hybrid microcapsule including: a shell that comprises one or more biocompatible material, exosomes contained within the microcapsule and one or more therapeutic cells encapsulated within the microcapsule, wherein the therapeutic cells are capable of releasing one or more therapeutic agent(s). Also disclosed are methods of making the hybrid microcapsule and methods of treating a subject including administering the hybrid microcapsule to the subject, wherein the therapeutic cells contained within the hybrid microcapsule release the one or more therapeutic agent(s) to the subject and wherein the hybrid microcapsule releases the exosomes to effectively attenuate an immune-based foreign body response (FBR).

### FIELD

The disclosure relates to hybrid microcapsules including a shell that comprises one or more biocompatible material, exosomes contained within the microcapsule, and one or more therapeutic cells encapsulated within the microcapsule, wherein the therapeutic cells are capable of releasing a therapeutic agent; methods making the hybrid microcapsules; and methods of treating a subject by administering the hybrid microcapsules.

### BACKGROUND

Transplantation of therapeutic cells has been examined as potential treatments for a variety of diseases, including, β-cells replacement therapies (Vegas, A. J. et al. Long-term glycemic control using polymer-encapsulated human stem cell-derived beta cells in immune-competent mice. Nat. Med. 22, 306-311 (2016)), bone marrow transplantation (Mao, A. S. et al. Programmable microencapsulation for enhanced mesenchymal stem cell persistence and immunomodulation. Proc. Natl Acad. Sci. USA 116, 15392 (2019)), and Parkinson's disease (Kojima, R. et al. Designer exosomes produced by implanted cells intracerebrally deliver therapeutic cargo for Parkinson's disease treatment. Nat. Commun. 9, 1305 (2018); and Parmar, M., Grealish, S. & Henchcliffe, C. The future of stem cell therapies for Parkinson disease. Nat. Rev. Neurosci. 21, 103-115 (2020)). Engineerability of cells and their responsiveness to the environmental cues make them living factories that deliver therapeutic agents with appropriate physiological dosing on demand. Yet, the promise of cell-based therapies is hampered by the challenges of achieving safe and effective long-term engraftment. Transplanted cells can elicit a strong immune response, especially if they originate from nonsyngeneic sources. Administration of immunosuppressive regimens (i.e., the non-steroidal anti-inflammatory agents) has been proposed to mute such immune responses, however, this approach may lead to detrimental side-effects including hepatocellular, cardiac or renal toxicities (Wehling, M. Non-steroidal anti-inflammatory drug use in chronic pain conditions with special emphasis on the elderly and patients with relevant comorbidities: management and mitigation of risks and adverse effects. Eur. J. Clin. Pharmacol. 70, 1159-1172 (2014)), gastrointestinal ulceration, bleeding, and microbial dysbiosis (Srinivasan, A. & De Cruz, P. Review article: a practical approach to the clinical management of NSAID enteropathy. Scand. J. Gastroenterol. 52, 941-947 (2017); and Tekin, Z. et al. Outcomes of pancreatic islet allotransplantation using the Edmonton protocol at the University of Chicago. Transpl. Direct 2, e105-e105 (2016)).

One notable example of therapeutic cell transplantation is the pancreatic islet transplantation to treat Type 1 diabetes (T1D), which has stimulated ˜50 years of research and clinical trials. Human trials on islet transplantation initiated with the Edmonton protocol, suggesting >5 years efficacy in some cases (Shapiro, A. M. J. et al. Islet transplantation in seven patients with Type 1 diabetes mellitus using a glucocorticoid-free immunosuppressive regimen. N. Engl. J. Med. 343, 230-238 (2000); and Shapiro, A. M. J. et al. International trial of the edmonton protocol for islet transplantation. N. Engl. J. Med. 355, 1318-1330 (2006)). However, adverse events through daily administration of immunosuppressive regimen as well as lack of allogeneic cell donors further compromised the clinical practice of Edmonton protocol (Tekin, Z. et al. Outcomes of pancreatic islet allotransplantation using the edmonton protocol at the University of Chicago. Transpl. Direct 2, e105-e105 (2016)). Encapsulation of islets within a protective biomaterial has been considered to eliminate this need for chronic immunosuppression (Desai, T. & Shea, L. D. Advances in islet encapsulation technologies. Nat. Rev. Drug Discov. 16, 338 (2016)). Dating back to the 1980s, islet transplantation within alginate microcapsules was found to prolong the glycemic correction in diabetic rodents (Franklin Lim, F. & Sun, A. M. Microencapsulated islets as bioartificial endocrine pancreas. Science 210, 908-910 (1980)). However, limited therapeutic efficacy and transient glycemic control have been reported in follow-up human trials of islet transplantation within alginate microcapsules (Desai, T. & Shea, L. D. Advances in islet encapsulation technologies. Nat. Rev. Drug Discov. 16, 338 (2016); Tuch, B. E. et al. Safety and viability of microencapsulated human islets transplanted into diabetic humans. Diabetes Care 32, 1887 (2009); Basta, G. et al. Long-term metabolic and immunological follow-up of nonimmunosuppressed patients with Type 1 diabetes treated with microencapsulated islet allografts. Diabetes Care 34, 2406 (2011); and Orive, G. et al. Engineering a clinically translatable bioartificial pancreas to treat type I diabetes. Trend. Biotechnol. 36, 445-456 (2018)). This suggests the restricted functionality of transplants through islet death and/or loss of mass transfer inwards and outwards of microcapsules. The main reasons behind such graft failure are likely to be the islet necrosis (due to the lack of nutrients and oxygen accessibility within the microcapsules) (Evron, Y. et al. Long-term viability and function of transplanted islets macroencapsulated at high density are achieved by enhanced oxygen supply. Sci. Rep. 8, 6508 (2018)), as well as immune-mediated pericapsular growth and fibrosis (Doloff, J. C. et al. Colony stimulating factor-1 receptor is a central component of the foreign body response to biomaterial implants in rodents and nonhuman primates. Nat. Mater. 16, 671 (2017); Bochenek, M. A. et al. Alginate encapsulation as long-term immune protection of allogeneic pancreatic islet cells transplanted into the omental bursa of macaques. Nat. Biomed. Eng. 2, 810-821 (2018); Farah, S. et al. Long-term implant fibrosis prevention in rodents and nonhuman primates using crystallized drug formulations. Nat. Mater. 18, 892-904 (2019); de Vos, P., Hamel, A. F. & Tatarkiewicz, K. Considerations for successful transplantation of encapsulated pancreatic islets. Diabetologia 45, 159-173 (2002); and Vaithilingam, V. & Tuch, B. E. Islet transplantation and encapsulation: an update on recent developments. Rev. Diabet. Stud. 8, 51 (2011)). The latter is also known as foreign body response (FBR), which creates considerable discomfort for patients and a variety of health complications (Mohammadi, M. R., Luong, J. C., Kim, G. G., Lau, H. & Lakey, J. R. T. in Handbook of Tissue Engineering Scaffolds, Vol. 1 (eds Mozafari, M., Sefat, F. & Atala, A.) (Woodhead Publishing, 2019); Swanson, E. Analysis of US Food and Drug Administration breast implant postapproval studies finding an increased risk of diseases and cancer: why the conclusions are unreliable. Ann. Plast. Surg. 82, 253-254 (2019); and Headon, H., Kasem, A. & Mokbel, K. Capsular contracture after breast augmentation: an update for clinical practice. Arch. Plast. Surg. 42, 532-543 (2015)).

Preventing the transplantation-led inflammatory response reduces pericapsular overgrowth and fibrosis. Many studies have demonstrated that islet transplantation within immunomodulator or immune-insulator microcapsules provides longterm euglycemia in immunocompetent diabetic rodents (Vegas, A. J. et al. Long-term glycemic control using polymer-encapsulated human stem cell-derived beta cells in immune-competent mice. Nat. Med. 22, 306-311 (2016); Farah, S. et al. Long-term implant fibrosis prevention in rodents and nonhuman primates using crystallized drug formulations. Nat. Mater. 18, 892-904 (2019); Vegas, A. J. et al. Combinatorial hydrogel library enables identification of materials that mitigate the foreign body response in primates. Nat. Biotechnol. 34, 345 (2016); Evron, Y. et al. Long-term viability and function of transplanted islets macroencapsulated at high density are achieved by enhanced oxygen supply. Sci. Rep. 8, 6508 (2018); and Alagpulinsa, D. A. et al. Alginate-microencapsulation of human stem cell-derived (3 cells with CXCL12 prolongs their survival and function in immunocompetent mice without systemic immunosuppression. Am. J. Transplant. 19, 1930-1940 (2019)). Various strategies have been employed to modulate and/or mute the local immune response against implants, including the surface-bound immunomodulatory ligands25, anti-biofouling surface modification (Vegas, A. J. et al. Combinatorial hydrogel library enables identification of materials that mitigate the foreign body response in primates. Nat. Biotechnol. 34, 345 (2016); Liu, Q. et al. Zwitterionically modified alginates mitigate cellular overgrowth for cell encapsulation. Nat. Commun. 10, 5262 (2019); and Spasojevic, M. et al. Reduction of the inflammatory responses against alginatepoly-L-lysine microcapsules by anti-biofouling surfaces of PEG-b-PLL deblock copolymers. PLoS ONE 9, e109837 (2014)), and controlled release of anti-inflammatory agents. The controlled-release (or drug eluting) biomaterials could hold and release variety of anti-inflammatory and/or immunomodulatory molecules overtime (e.g., dexamethasone (Vacanti, N. M. et al. Localized delivery of dexamethasone from electrospun fibers reduces the foreign body response. Biomacromolecules 13, 3031-3038 (2012)), IL-4 (Hachim, D., LoPresti, S. T., Yates, C. C. & Brown, B. N. Shifts in macrophage phenotype at the biomaterial interface via IL-4 eluting coatings are associated with improved implant integration. Biomaterials 112, 95-107 (2017)), CSF1R inhibitor (Farah, S. et al. Long-term implant fibrosis prevention in rodents and nonhuman primates using crystallized drug formulations. Nat. Mater. 18, 892-904 (2019)), and CXCL12 (Alagpulinsa, D. A. et al. Alginate-microencapsulation of human stem cell-derived (3 cells with CXCL12 prolongs their survival and function in immunocompetent mice without systemic immunosuppression. Am. J. Transplant. 19, 1930-1940 (2019))). There are two main possible drawbacks with many of these molecular target inhibitors. The first issue is the potential side effects associated with these agents. For instance, CSF1R inhibitors can elicit fatigue/asthenia, edema (Cannarile, M. A. et al. Colony-stimulating factor 1 receptor (CSF1R) inhibitors in cancer therapy. J. Immunother. Cancer 5, 53 (2017)), and nonreversible grade 3 deafness (Papadopoulos, K. P. et al. First-in-human study of AMG 820, a monoclonal anti-colony-stimulating factor 1 receptor antibody, in patients with advanced solid tumors. Clin. Cancer Res. 23, 5703-5710 (2017)), and CXCL12 causes toxicity in cerebrocortical neurons (Sanchez, A. B. et al. CXCL12-induced neurotoxicity critically depends on NMDA receptor-gated and 1-type Ca2+ channels upstream of p38 MAPK. J. Neuroinflammation 13, 252 (2016)). Other molecular targets such as TNFα inhibitors and anti-TGFβ compounds are also linked to a variety of complications in clinical trials (Lin, J. T. et al. TNFα blockade in human diseases: an overview of efficacy and safety. Clin. Immunol. 126, 121-136 (2008); and Walton, K. L., Johnson, K. E. & Harrison, C. A. Targeting TGF-β mediated SMAD signaling for the prevention of fibrosis. Front Pharm. 8, 461-461 (2017)). The second challenge with molecular inhibitors lies in their inability to regulate a multitude of inflammatory pathways involved in the immune response against biomaterials transplants, including NFκB (Amer, L. D. et al. Inflammation via myeloid differentiation primary response gene 88 signaling mediates the fibrotic response to implantable synthetic poly (ethylene glycol) hydrogels. Acta Biomater. 100, 105-117 (2019); Yang, D. & Jones, K. S. Effect of alginate on innate immune activation of macrophages. J. Biomed. Mater. Res. Part A 90A, 411-418 (2009) and Lawlor, C. et al. Treatment of Mycobacterium tuberculosis-infected macrophages with poly(lactic-co-glycolic acid) microparticles drives NFκB and autophagy dependent bacillary killing. PLoS ONE 11, e0149167 (2016)), CSF1R (Doloff, J. C. et al. Colony stimulating factor-1 receptor is a central component of the foreign body response to biomaterial implants in rodents and nonhuman primates. Nat. Mater. 16, 671 (2017); and Farah, S. et al. Long-term implant fibrosis prevention in rodents and nonhuman primates using crystallized drug formulations. Nat. Mater. 18, 892-904 (2019)), and JAK/STAT (Moore, L. B. & Kyriakides, T. R. in Immune Responses to Biosurfaces (eds Lambris, J. D., Ekdahl, K. N., Ricklin, D. & Nilsson, B.) (Springer International Publishing, 2015)) pathways. Thus, it is speculated that the controlled release of agents that regulate multiple inflammatory pathways may better mute the inflammatory response against implants compared to agents that interfere with single targets.

In this context, mesenchymal stromal cells (MSCs, also named as medicinal signaling cells) are recognized to regulate variety of inflammatory pathways including NFκB (Su, V. Y.-F., Lin, C.-S., Hung, S.-C. & Yang, K.-Y. Mesenchymal stem cell-conditioned medium induces neutrophil apoptosis associated with inhibition of the NF-κB pathway in endotoxin-induced acute lung injury. Int. J. Mol. Sci. 20, 2208 (2019)), JAK/STAT (Vigo, T. et al. IFN-γ orchestrates mesenchymal stem cell plasticity through the signal transducer and activator of transcription 1 and 3 and mammalian target of rapamycin pathways. J. Allergy Clin. Immunol. 139, 1667-1676 (2017)), MyD88 (Chen, C.-P., Tsai, P.-S. & Huang, C.-J. Antiinflammation effect of human placental multipotent mesenchymal stromal cells is mediated by prostaglandin E2 via a myeloid differentiation primary response gene 88-dependent pathway. Anesthesiology 117, 568-579 (2012)), and PI3K/AKT (Riazifar, M. et al. Stem cell-derived exosomes as nanotherapeutics for autoimmune and neurodegenerative disorders. ACS Nano 13, 6670-6688 (2019)). The current paradigm of MSC treatment is through paracrine factor, which could partly be attributed to MSC-derived exosomes (XOs) (Yin, J. Q., Zhu, J. & Ankrum, J. A. Manufacturing of primed mesenchymal stromal cells for therapy. Nat. Biomed. Eng. 3, 90-104 (2019); Riazifar, M., Pone, E. J., Lötvall, J. & Zhao, W. Stem cell extracellular vesicles: extended messages of regeneration. Annu. Rev. Pharmacol. Toxicol. 57, 125-154 (2017)). While detailed mechanisms behind immunomodulatory effects of XOs are not yet fully understood, they have been recognized for their capability to regulate the function of multiple immune cell types including macrophages (Lankford, K. L. et al. Intravenously delivered mesenchymal stem cell-derived exosomes target M2-type macrophages in the injured spinal cord. PLoS ONE 13, e0190358 (2018)), NK cells (Fan, Y. et al. Human fetal liver mesenchymal stem cell-derived exosomes impair natural killer cell function. Stem Cells Dev. 28, 44-55 (2018); and Burrello, J. et al. Stem cell-derived extracellular vesicles and immunomodulation. Front. Cell Develop. Biol. 4, 83 (2016)), B cells (Khare, D. et al. Mesenchymal stromal cell-derived exosomes affect mRNA expression and function of B-lymphocytes. Front. Immunol. 9, 3053-3053 (2018); and Carreras-Planella, L., Monguio-Tortajada, M., Borràs, F. E. & Franquesa, M. Immunomodulatory effect of MSC on B cells is independent of secreted extracellular vesicles. Front. Immunol. 10, 1288 (2019)), and T lymphocytes (Shigemoto-Kuroda, T. et al. MSC-derived extracellular vesicles attenuate immune responses in two autoimmune murine models: Type 1 diabetes and uveoretinitis. Stem Cell Rep. 8, 1214-1225 (2017)). We thus hypothesized that co-transplantation of XOs within alginate microcapsules (AlgXO) would alleviate the FBR upon implantation. Upon blocking this inflammation, we next hypothesized that transplantation of rat islets within AlgXO would prolong the function of transplanted islets in immunocompetent streptozotocin-induced diabetic mice.

### SUMMARY OF THE INVENTION

Some examples relate to a hybrid microcapsule including:
(a) a shell that comprises one or more biocompatible material,
(b) exosomes contained within the microcapsule, and
(c) one or more therapeutic cells encapsulated within the microcapsule, wherein the therapeutic cells are capable of releasing one or more therapeutic agent(s).

In some examples, the one or more biocompatible material is a natural material selected from the group consisting of alginate, pectin, agarose, collagen and hyaluronic acid or a synthetic material selected from the group consisting of poly(ethylene glycol) (PEG), 2-hydroxyethyl methacrylate (HEMA) and poly(lactic-co-glycolic acid) (PLGA).

In some examples, the one or more biocompatible material includes an alginate or a derivative thereof.

In some examples, the alginate or derivative thereof is a cross-linked ultrapure alginate.

In some examples, the outer surface of the shell is hydrophilic and resistant to protein binding.

In some examples, the exosomes are derived from mesenchymal stem cells (MSCs).

In some examples, the mesenchymal stem cells are umbilical cord mesenchymal stem cells.

In some examples, the umbilical cord mesenchymal stem cells are human umbilical cord mesenchymal stem cells.

In some examples, the exosomes have a particle diameter of from 10-500 nm.

In some examples, the exosomes have a particle diameter of from 20-200 nm.

In some examples, the microcapsule includes 1×10⁵-1×10⁸ exosomes within the microcapsule.

In some examples, the one or more therapeutic cells include pancreatic islets.

In some examples, the microcapsule includes 1-10 islet equivalent (IEQ) cells.

Some examples relate to a method of making the hybrid microcapsule according to claim 1 including:
(a) isolating exosomes from mesenchymal stem cells (MSCs),
(b) obtaining therapeutic cells that are capable of releasing one or more therapeutic agent(s), and
(c) incorporating the exosomes and the therapeutic cells into a microcapsule.

In some examples of the method, the microcapsule is an alginate microcapsule.

In some examples of the method, the MSCs are umbilical cord derived MSCs (UC-MSCs).

Some examples relate to a method of treating a subject including administering the hybrid microcapsule disclosed herein to the subject, wherein the therapeutic cells contained within the hybrid microcapsule release the therapeutic agent to the subject and wherein the hybrid microcapsule releases the exosomes to effectively attenuate an immune-based foreign body response (FBR) and enhance the viability of the encapsulated therapeutic cells.

In some examples of treating a subject, the therapeutic cells are pancreatic islet cells and wherein the subject is treated for Type 1 diabetes.

Some examples relate to a method of attenuating an immune response to a microcapsule in a subject including administering a microcapsule comprising exosomes contained within the microcapsule to the subject, wherein the exosomes are released from the microcapsule and wherein, upon release, the exosomes suppress a local immune microenvironment and effectively attenuate the immune response.

In some examples, the immune response to the microcapsule is an immune-based foreign body response (FBR) to biomaterials in the microcapsule.

Some examples relate to a method of replacing the immunosuppressive regimen before or during the islet transplantation.

In some examples, exosomes replace the immunosuppressive regimen.

### BRIEF DESCRIPTION OF THE DRAWINGS

FIG. 1. Long-term normoglycemia in immunocompetent diabetic mouse models with 5000 IEQ rat islets encapsulated in alginate (line with circles) and exosome encapsulated (line with squares).

FIG. 2. Islet xenotransplants within AlgXO reverse hyperglycemia in diabetic immunocompetent mice. (a) Non-fasting blood glucose levels in C57/BL6 STZ-induced diabetic mice (n=5 mice) shows that transplantation of 1500 IEQ rat islets within AlgXO provided euglycemia in diabetic mice for >170 days, whereas the CTRL microcapsules failed in <1 month. To further confirm that the glycemic correction is merely due to transplants and not pancreatic regeneration in STZ-induced diabetic mice, we washed the i.p. cavity of mice and removed the explants after 105 days of transplantation (n=2 mice). Within 18 h of graft removal, mice blood glucose elevated and remained hyperglycemic for the rest of their lifetime (dashed line). Separate i.p. transplantation of islets within CTRL microcapsules and XOs provided normoglycemia for ˜70 days (black line, n=4 mice). (b) We further tested the efficacy of AlgXO transplants in response to oral glucose tolerance test (OGTT). One month after transplantation, similar to STZ mice (n=6 mice), CTRL microcapsules failed to regulate the glucose levels (n=4 mice), whereas AlgXO transplants successfully reversed hyperglycemia event induced by glucose challenge (n=6 mice), with similar trend as non-diabetic controls. (c) The average time to reach normoglycemia after an OGTT for non-diabetic mice was 65±27 min and for mice with AlgXO transplants was 103±32 min (n=6 mice). (d) After 1 month, both CTRL and AlgXO (from 1500 IEQ group) transplants were removed through washing the i.p. cavity. Next, microcapsules were analyzed for the immune infiltration (also known as pericapsular cell growth) with laser-scanning confocal microscopy. Some cells were CD11b+ and some of the CD11b+ cells were expressing MHCII biomarker. All the collected CTRL microcapsules were found to have pericapsular cells attached to the surface, while the percentage of AlgXO transplants with pericapsular growth was 9.4%±3.6%, which was significantly lower than CTRL transplants (p<0.0001). Scale bars are 200 μm for the dark field and 100 μm for the florescent channels. (e) The pericapsular cytokine and chemokines present released in the pericapsular area of implants. Results are mean±SD, and statistical significance is calculated through unpaired t-test with Welch's correction. 1: STZ injection; 2: Diabetes induction period; 3: Transplantation; 4: Graft removal.

FIG. 3. (a) Study design to compare and quantify the inflammatory response against AlgXO and CTRL microcapsules. A two weeks study was performed since this timeframe is suitable for resolving and reflecting both innate and adaptive immune system as well as fibrotic responses to implanted materials in C57/BL6 mice. Blood was collected on days 7 and 14 for immunocytes and inflammatory cytokines analyses (n=4). (b) Two weeks after implantation, MCP-1 chemokine was 3.7-fold less in the bloodstream of mice that had received AlgXO versus the ones transplanted with CTRL. (c) CD45+CD11b+Ly6c^{high}Ly6G_{med} inflammatory monocytes were significantly lower (p=0.002) in the bloodstream of mice transplanted with AlgXO compared to CTRL (n=3). (d) While captured images from explants and their BF microscopy are similar, sections and scanning electron micrographs from two weeks explants show different immune-environment around microcapsules. White arrows show the localization of grafts and yellow arrows point to the cells infiltrated around microcapsules. (e) and (f) Fixed fibrotic tissues were later sectioned and stained for sub-populations of immunocytes. Normalized areas of DAPI, CD68, and MHCII around explant microenvironments of AlgXO were significantly lower than CTRL microcapsules (n=4). (g) Total cells of fibrotic tissues were isolated and stained for flow cytometry analyses of immunocytes subpopulation. tSNE plots further demonstrates the different immune-environment around AlgXO and CTRL explants. We further conducted a query on a subpopulation that is present on CTRL but absent in AlgXO immune-environment. This subpopulation is CD45+CD11b+CD19+MHCII+CD3−Ly6C−, which is likely to be the memory B cells sob-population. Statistical significance is calculated through unpaired t-test with Welch's correction.

FIG. 4. AlgXO reduces FBR partly due to releasing XOs in a controlled fashion. (a) a non-significant difference between AlgXO's and CTRL's captive bubble contact angle was observed (i.e., 156.3°±3.8° for AlgXO versus 150.2°±4.9° for CTRL). (b) IgG protein adsorption to the surface of AlgXO and CTRL microcapsules (n=3). Microscale mechanical testing was performed for (c) AlgXO and CTRL. (d) Stress-Strain curve of AlgXO vs. CTRL microcapsules graphed based on the force-displacement data. Elastic modulus was not significantly different (p=0.268) between AlgXO (104.7±61.4 kPa) and CTRL (57.8±14.9 kPa). (e) to investigate the release of encapsulated exosomes, scanning electron microscopy was conducted on air-dried microcapsules, demonstrating surface pores in 50-200 nm size scale (scale=1 μm), and encapsulation of vesicles within AlgXO. (f) schematic representation of our hypothesized model, where XOs release from AlgXO microcapsules overtime. (g) AlgXO releases XOs in a control fashioned in vitro. Release profile reaches a threshold within a week. (h) diffusion of nanoparticles with diameters 50, 100, and 150 nm (which are chosen due to the size ranges of XOs). Particles number vs. time vs. distance from microcapsule center (d) are graphed in the percentage heatmap diagrams. Bottom panels show the color map of spatiotemporal diffusion rate of XOs at t=0, 50, 300, and 600 s upon initiation of diffusion. Expectedly, the smaller the size, the higher the diffusion rate. In addition, 600 s after the onset of diffusion, the concentration of 50 nm particles at the center of the microcapsules dropped 20%, while for 100 nm and 150 nm, no significant decrease was obtained at the center of the microcapsules. Statistical significance is calculated through unpaired t-test with Welch's correction.

FIG. 5. XOs suppress the proliferation of splenocytes and CD3+ T cells and reduce the production of inflammatory cytokines from LPS stimulated macrophages (a) Schematic figure showing the experimental procedure. CFSE labeled splenocytes and CD3+ T cells were co-cultured with plate bound anti-CD3 and soluble CD28 in the presence of absence of 20 and 200 μg/mL of XOs. Upon 4 days co-culture, cells were analyzed using flow cytometry. (b) Splenocyte counts for CD3/CD28 activated cells were 9603±871, and addition of 20 and 200 μg/mL XOs reduced the counts to 1253±1038 (n=4, p<0.0001) and 1570±1010 (n=4, p<0.0001), respectively. (c) In the co-cultures of CD3+ cells with CD3/CD28 antibodies, CD4+ counts for CD3/CD28 activated T cells was 5217±378. Addition of 20 and 200 μg/mL XOs reduced the counts to 3889±2081 (n=4, p=0.0031) and 4387±1397 (n=4, p=0.0057), respectively. (d) In the co-cultures of CD3+ cells with CD3/CD28 antibodies, CD8+ counts for CD3/CD28 activated T cells was 2700±252. Addition of 20 and 200 μg/mL XOs reduced the counts to 1503±784 (n=4, p=0.0018) and 1766±628 (n=4, p=0.0002), respectively. (e) Addition of XOs to the co-cultures of murine macrophages reduce the secretion of inflammatory cytokines (G-CSF, IFNγ, IL-6, LIF, LIX, MIP-2, RANTES) in a dose dependent manner (n=4). Statistical significance is calculated through unpaired t-test with Welch's correction.

FIG. 6. XOs suppress human peripheral blood mononuclear cells and macrophages. (a) Human Peripheral Blood Mononuclear Cells (PBMCs) were activated with bead-bound CD3/CD28 antibodies in the presence and absence of XOs. (b) Addition of 20 μg/mL and 200 μg/mL XOs reduced the count of activated PBMCs from 24002±6762 to 2342±910 (n=3; p=0.029) and to 2102±1121 (n=3; p=0.027), respectively. To gain more insight into the XOs mechanism of action, cytokine production was evaluated in the PBMCs culture. Addition of XOs reduced the IL-6, TNFα, IL-12p70, and IL-22 production from activated PBMCs. (c) In the co-cultures of anti-CD3/CD28 activated PBMCs, addition of XOs reduce IL-2, IL-6, IL-10, IL-12p70, IL-22, and TNFα (n=3). (d) XOs suppressed the LPS mediated human macrophages activation. LPS activated NFκB pathway in THP-1 macrophages, and addition of 200 μg/mL of XOs reduces NFκB activation of both 10 ng/mL LPS (n=4, p=0.044) and 100 ng/mL LPS (n=4, p=0.004) activated THP-1 macrophages. 20 μg/mL of XOs was not enough to interfere with the NFκB activation. XOs influenced the NFκB activation of non-activated THP-1 cells. Addition of 20 μg/mL XOs upregulated the NFκB activity in THP-1 cells from 109±17 to 203±20 (n=4, p=0.0117). Furthermore, addition of 200 μg/mL XOs upregulated the NFκB activity in THP-1 cells from 109±17 to 215±23 (n=4, p=0.0105). Statistical significance is calculated by unpaired t-test with Welch's correction.

FIG. 7. Umbilical Cord Derived Stem Cells (MSCs) and their secreted XOs characterization. (a) Cells were characterized for their surface markers, showing the low expression of Stro-1, high expression CD90/Thy1, CD146/MCAM, CD105/Endoglin, CD166, CD44 while cells are negative for CD19, CD45 and CD106. Cells were then cultured as described in the Materials & Methods section, and XOs were isolated. (b) Isolated XOs were then characterized using stablished biomarkers using Western blotting. XOs were positive for CD63, Galectin 1, TSG101, HSP70, Hsp70, and negative for and endoplasmic reticulum marker Calnexin. (c) XOs possess spherical shape with the 105±48 nm as an average size for maximum quantity of vesicles, based on NTA analyses. It should be noted that in average, 1.7×10¹²±7.6×10¹⁰ XOs/mL were isolated from ˜150 to 190 million cultured MSCs in 100% confluency. (d) Flow cytometry analysis of TGFβ, PD-L1, and MHCII expression on XOs bound to anti-CD63-coated beads. Statistical significance is calculated through unpaired t-test with Welch's correction.

FIG. 8. (a) Freeze-Fractured Scanning Electron Microscopy of an AlgXO microcapsule, showing the encapsulated XOs within the microcapsules (Scale=100 μm). (b) total number of exosome encapsulated within ˜1000 AlgXO microcapsules is 5.43×10⁹±4.84×10⁹ using NTA analysis. (n=4 separate preparation)

FIG. 9. EDTA dissolves alginate microcapsules. CTRL microcapsules (n=100) were dissolved in 5 or 10 mM EDTA, and microscopic images were taken in 1 min intervals using EVOS imaging system microscope.

FIG. 10. Islets quality control. After each islet isolation, we ran quality control measurements. (a) DTZ staining to quantify the islet purity and count (947±137 IEQ). (b) Glucose Stimulation Insulin Release (GSIR) test is run to validate the functionality of the isolated islets. Encapsulated (c) CTRL microcapsules and (d) AlgXO microcapsules

FIG. 11. Transplantation of AlgXO microcapsules without islets failed to reverse hyperglycemia in STZ mice. Empty (without islets) AlgXO microcapsules could not reverse the hyperglycemia in diabetic mice.

FIG. 12. Polynomial regressions onto glucose challenge response. (a) Polynomials with degree 5 were assigned to the OGTT curve of every individual mice of a non-diabetic group and (b) AlgXO transplanted group (1500 IEQ). Small circles show the raw OGTT data and lines represent the assigned polynomial. Dashed line demonstrates the normoglycemic criterion (i.e., blood glucose <200 mg/mL).

FIG. 13. Dose study of islets xenotransplantation (i.e. 500 or 5000 IEQ islets) in immunocompetent STZ mice. (a) In higher islet dosage (5000 IEQ), CTRL transplants failed to consistently reverse hyperglycemia in C57/BL6 STZ mice. However, AlgXO transplants reversed hyperglycemia for ˜80 days. (b) We further tested the efficacy of AlgXO transplants in response to oral glucose tolerance test (OGTT). One month after transplantation, AlgXO transplants successfully reversed hyperglycemia event induced by glucose challenge with a similar trend as non-diabetic mice. (c) Polynomials with degree 5 were assigned to the OGTT curve of every individual mice and equations were solved to find the average time needed for the mice blood glucose to reach 200 mg/dL after an OGTT. (d) The average time to reach normoglycemia (i.e., 200 mg/dL) after an OGTT was 112±32 minutes for mice transplanted with 5000 IEQ islet encapsulated in AlgXO. The average time to reach normoglycemia for non-diabetic mice was 67±26 minutes. This suggest a slight delay in glucose response of mice received AlgXO transplants versus non-diabetic mice (p=0.08). (e) Mice received CTRL transplants with 5000 IEQ islets had low survivals, where 6 of 10 mice died within a day of transplantation, while only 1/7 mice receiving AlgXO transplants with 5000 IEQ islets died within one day of transplantation and 5 others remained alive till the end of the study (p=0.0018, Long-rank (Mantel-Cox) test). (f) Mice that received AlgXO transplants with 5000 IEQ islets remained normoglycemic for 75±7 days, while this duration for CTRL transplanted mice was 9±7 days after transplantation. (g) Lower dose islets (500 IEQ) was ineffective in euglycemic induction neither within CTRL nor AlgXO microcapsules. On the diagram, 1 shows the STZ induction, 2 shows the time for diabetes progression, and 3 shows the transplantation timepoints. On the diagram, 1 shows the STZ induction, 2 shows the time for diabetes progression, and 3 shows the transplantation timepoints. Statistical significance is calculated through unpaired t-test with Welch's correction.

FIG. 14. XOs enhance the viability of naked and encapsulated rat islets. (a) Addition of 20 μg/mL and 200 127 μg/mL XOs to the islet cultures significantly enhances the viability of islets after 5 and 7 days of culture. It should be noted that 128 viability was measured using Calcein AM (live cells) and propidium iodide (dead cells) staining. (b) Starting from 3 days of islet 129 encapsulation, AlgXO enhances the viability of encapsulated islets within the first week of encapsulation. (c) TUNEL assay 130 demonstrated that after 1 month of transplantation, the TUNEL positive area of islets transplanted within AlgXO (1.02%±0.32%) 131 is higher (n=5, p=0.0256) compared to CTRL (6.44%±1.59%). Statistical significance is calculated through unpaired t-test 132 with Welch's correction.

FIG. 15. Blood cytokines analyses of mice received AlgXO or CTRL microcapsules. Mice serum was harvested from mice at days 7 and 14 after transplantation, showing no significant difference among groups. One-way ANOVA was conducted to measure the statistical difference. Wiled Type (WT) mice was also added to the groups as a negative control (n=4, statistical significance is calculated through unpaired t-test with Welch's correction).

FIG. 16. Vasculature present in the fibrotic tissue around AlgXO. (a) Pictures from subcutaneous explants show presence of blood vessels in AlgXO fibrotic microenvironment. (b) Flow cytometry analyses shows the presence of higher CD45+ cells (p<0.0001) harvested from AlgXO fibrotic tissues (33.1%±8.0%) compared to control (83.0%±12.8%). (c) αSMA (markers of blood vessels) were absent in CTRL fibrotic tissues compared to (d) AlgXO. Statistical significance is calculated through unpaired t-test with Welch's correction.

FIG. 17. The percentage of (a) B and (b) T cells presence in the fibrotic Tissues around AlgXO and CTRL. (n=4). Statistical significance is calculated through unpaired t-test with Welch's correction.

FIG. 18. Flow cytometry analyses of lavage around microcapsules show distinct immunocytes population around AlgXO and CTRL microcapsules. (a and b) Flow cytometry analyses demonstrates the total CD45+ population present around AlgXO is less than CTRL microcapsules. Similar trend was observed for CD11b+, CD11b+MHCII+, and CD11b+MHCII-CD206+ sub-populations. (c) tSNE plots demonstrates the different cell environment present in the lavage collected from surrounding non/low adherent cells around AlgXO and CTRL explants. (d) Two sub-populations were then analyzed for immune markers. Cells in Query 1 (gated on specific sub population present in CTRL but not in AlgXO) was CD45+CD11b+CD3-CD19−MHCII−Ly6C−Ly6G−, which is likely to be dendritic cells. Cells in Query 2 (gated on specific sub population present in AlgXO but not in CTRL) was CD45−CD11b-CD3-CD19−MHCII−Ly6C−Ly6G−, which is likely to be neither from myeloid or lymphoid origin. (n=4, statistical significance is calculated through unpaired t-test with Welch's correction)

FIG. 19. Subcutaneous transplantation of islets encapsulated in either CTRL and AlgXO. The (a) glucose and (b) body weights of STZed mice were tracked for a month, and there was no significant improvement in the glycemic control in any of the groups.

FIG. 20. Simulated controlled release of particles with 10, 50, 100, 200, or 500 nm of diameters. At t >0, particles with diameter <200 nm show diffusion profiles, where smaller particles diffuse faster. Particles with diameter of 500 nm do not show diffusion out of microcapsules at least for 600 s.

FIG. 21. XOs effect on the production of cytokines from 10 ng/mL LPS (TLR4 agonist) stimulated macrophages. Statistical significance is calculated through unpaired t-test with Welch's correction (n=4).

FIG. 22. Cytokines analyses from co-cultures of human activated PBMCs. PBMCs were activated with bead-bound CD3/CD28 antibodies in the presence and absence of XOs. XOs in both 20 and 200 μg/mL concentrations slightly influenced the production of IL-1(3, IL-23, IFNγ, and IDO (n=4). Statistical significance is calculated through unpaired t-test with Welch's correction.

### DETAILED DESCRIPTION

Type 1 Diabetes (T1D) has a significant burden on US economic healthcare expenditure. In 2009, the annual institutional care cost for diabetic patients was $10 billion, of which $4.4 billion was spent on care for T1D patients (Dail, T. et al. 2009 Population Health Management 12(2): 103-110). 1.3 million adults and children in the US are estimated to suffer from T1D, and the number is anticipated to exceed 5 million by 2050 (Imperatore, G. et al. 2012 Diabetes Care 35(12): 2515-2520). Current therapy includes direct injection of insulin to patients leading to patient incompliance and discomfort. The publication of Edmonton protocol in 2000 was a breakthrough in clinical islet transplantation and improved the frequency of patients that remained insulin independent by employing a series of immunosuppressants (Shapiro, A. M. et al. 2000 New England Journal of Medicine 343: 230-238). Islet Anoikis (detachment of islets from extracellular matrix) as well as instant blood mediated inflammatory reaction (IBMIR) challenges led the researchers to implement a protective biomaterial around the islets (Coronel M. M. et al. 2013 Current Opinion in Biotechnology 24(5): 900-908). Even though biomaterial encapsulation inhibits the infiltration of leukocytes into the islets' vicinity, in most cases innate immunity responds to implants by forming the fibrotic capsule at the interface of biomaterials and the surrounding niche (Doloff, J. C. et al. 2017 Nature Materials 16(6): 671-680). This fibrotic capsule leads to impairment in glucose-insulin transport, blood supply, and engraftment of the implanted materials. To address this, some studies have been devoted to find anti-fibrotic formulations (Vegas, A. J. et al. 2016 Nature Biotechnology 34(3): 345-352). Other studies focused on biological scaffolds using plasma and recombinant human thrombin (Berman, D. M. et al. 2016 Diabetes 65(5): 1350-1361). We sought a unique approach, using stem cell derived exosomes to be encapsulated within alginate microcapsules.

Here, we present an engineered biomimetic scaffold, where the scaffold is built by infiltration of endogenous cells and extracellular components. This scaffold is capable of immunoengineering exosomes overtime, where the surrounding immune myriad is reprogrammed. This concept is of significant importance in encapsulated islet transplantation treatment of patients with Type 1 diabetes. Often, the immune response against transplanted capsules creates a fibrotic tissue around the capsules, which limits the insulin-release capabilities of encapsulated islets. With this technology, encapsulated islets may last longer leading to better treatment for diabetic patients. Our current results have shown a 90 days delay in the graft rejection of transplanted islets as well as euglycemia. Our initial evaluation was performed using alginate as the biomaterial, but could be performed with other biomaterials.

### Bioencapsulation Materials

Both natural and synthetic polymers have been used for bioencapsulation. Natural polymers such as alginate, pectin, agarose, collagen and hyaluronic acid are abundant and biocompatible and can be used for bioencapsulation under mild conditions (Gasperini L, Mano J F, Reis R L. Natural polymers for the microencapsulation of cells. J R Soc Interface. 2014; 11(100):20140817). However, their product quality and characteristics can vary broadly among resources and batches. It is well known that a natural polymer's purity and composition, such as the guluronic and mannuronic acid ratio of alginate, highly influence the capsule's performance (Zhang W J, Li B G, Zhang C, Xie X H, Tang T T. Biocompatibility and membrane strength of C3H10T1/2 cell-loaded alginate-based microcapsules. Cytotherapy. 2008; 10(1):90-97; Orive G, Santos E, Poncelet D, et al. Cell encapsulation: technical and clinical advances. Trends Pharmacol Sci. 2015; 36(8):537-546; and Zhang, W. Encapsulation of transgenic cells for gene therapy, Gene Therapy: principles and challenges. Hashad, D., editor. InTech; InTech: Rijeka, Croatia; 2015.). Synthetic polymers such as poly(ethylene glycol) (PEG), 2-hydroxyethyl methacrylate (HEMA) and poly(lactic-co-glycolic acid) (PLGA) exhibit more consistent chemical compositions and molecular weights due to the minimized batch-to-batch variations (Zhang W, He X. Microencapsulating and banking living cells for cell-based medicine. J Healthc Eng. 2011; 2(4):427-446; Zhang, W. Encapsulation of transgenic cells for gene therapy, Gene Therapy: principles and challenges. Hashad, D., editor. InTech; InTech: Rijeka, Croatia; 2015; Santos E, Zarate J, Orive G, Hernandez R M, Pedraz J L. Biomaterials in cell microencapsulation. Adv Exp Med Biol. 2010; 670:5-21; and Olabisi R M. Cell microencapsulation with synthetic polymers. J Biomed Mater Res A. 2015; 103(2):846-859). When using synthetic polymers for bioencapsulation, unfavorable conditions may be required, such as exposure to UV light and nonphysiological pH and/or temperature conditions (Olabisi R M. Cell microencapsulation with synthetic polymers. J Biomed Mater Res A. 2015; 103(2):846-859).

Among the natural and synthetic polymers, alginate and PEG are two of the most commonly used bioencapsulation materials. Alginates, anionic biopolymers mainly extracted from seaweed, are linear polysaccharides (Bidarra S J, Barrias C C, Granja P L. Injectable alginate hydrogels for cell delivery in tissue engineering. Acta Biomater. 2014; 10(4):1646-1662). Alginates are composed of α-L-guluronic acid (G) and β-D-mannuronic acid (M) blocks. Formation of the divalent cation junctions—of GG-GG, MG-GG and MG-MG—between alginate molecules leads to the gelation of alginate (formation of the alginate hydrogel) (Zhang W, He X. Microencapsulating and banking living cells for cell-based medicine. J Healthc Eng. 2011; 2(4):427-446).

In general, alginate microcapsules may be coated with a polycation, such as poly-L-lysine or chitosan, to enhance stability and impart permselectivity and PEG to improve the biocompatibility for tissue-engineering applications (Zhang W, Zhao S, Rao W, et al. A novel core-shell microcapsule for encapsulation and 3d culture of embryonic stem cells. J Mater Chem B Mater Biol Med. 2013; 2013(7):1002-1009; Gattás-Asfura K, Valdes M, Celik E, Stabler C. Covalent layer-by-layer assembly of hyperbranched polymers on alginate microcapsules to impart stability and permselectivity. J Mater Chem B Mater Biol Med. 2014; 2(46):8208-8219; and Park H S, Kim J W, Lee S H, et al. Antifibrotic effect of rapamycin containing polyethylene glycol-coated alginate microcapsule in islet xenotransplantation. J Tissue Eng Regen Med. 2017 11(4):1274-1284). Alginate also exhibits excellent in vivo stability (Zanotti L, Sarukhan A, Dander E, et al. Encapsulated mesenchymal stem cells for in vivo immunomodulation. Leukemia. 2013; 27(2):500-503). However, multiple factors can influence alginate-based capsule stability after transplantation, such as the implantation site and capsule composition (Kollmer M, Appel A A, Somo S I, Brey E M. Long-term function of alginate-encapsulated islets. Tissue Eng Part B Rev. 2015; 22(1):34-46). Retrieval of live encapsulated porcine islets from a patient 9.5 years after xenotransplantation has been reported (Elliott R B, Escobar L, Tan P L, Muzina M, Zwain S, Buchanan C. Live encapsulated porcine islets from a Type 1 diabetic patient 9.5 yr after xenotransplantation. Xenotransplantation. 2007; 14(2):157-161).

PEG and its derivatives, e.g., poly(ethylene glycol) diacrylate [PEGDA], have been widely used in tissue engineering due to their biocompatibility and ability to be altered to physically mimic soft tissues (Olabisi R M, Lazard Z W, Franco C L, et al. Hydrogel microsphere encapsulation of a cell-based gene therapy system increases cell survival of injected cells, transgene expression, and bone volume in a model of heterotopic ossification. Tissue Eng Part A. 2010; 16(12):3727-3736; Mumaw J, Jordan E T, Sonnet C, et al. Rapid heterotrophic ossification with cryopreserved poly(ethylene glycol-) microencapsulated BMP2-expressing MSCs. Int J Biomater. 2012; 2012:861794). PEG is one of the few synthetic polymers that can be used for both microencapsulation and macroencapsulation (de Vos P, Lazarjani H A, Poncelet D, Faas M M. Polymers in cell encapsulation from an enveloped cell perspective. Adv Drug Deliv Rev. 2014; 67-68:15-34), and it has been extensively studied for the surface modification of scaffolds, such as vascular grafts, due to its nonimmunogenicity and nonantigenicity (Ren X, Feng Y, Guo J, et al. Surface modification and endothelialization of biomaterials as potential scaffolds for vascular tissue engineering applications. Chem Soc Rev. 2015; 44(15):5680-5742; Pramanik S, Ataollahi F, Pingguan-Murphy B, Oshkour A A, Osman N A. In vitro study of surface modified poly(ethylene glycol)-impregnated sintered bovine bone scaffolds on human fibroblast cells. Sci Rep. 2015; 5: 9806). There are different methods for preparing soft PEG gels, such as crosslinking via copper-free strain azide-alkyne cycloaddition (M Jonker A, A Bode S, H Kusters A, van Hest J C, Lowik D W. Soft PEG-Hydrogels with independently tunable stiffness and rgds-content for cell adhesion studies. Macromol Biosci. 2015; 15(10):1338-1347) and thiol-ene click chemistry (McKinnon D D, Kloxinb A M, Anseth K S. Synthetic hydrogel platform for three-dimensional culture of embryonic stem cell-derived motor neurons. Biomater Sci. 2013; 1(5):460-469). Microcapsules create uniform surfaces without rough edges. Lathuilère et al (Lathuilière A, Cosson S, Lutolf M P, Schneider B L, Aebischer P. A high-capacity cell macroencapsulation system supporting the long-term survival of genetically engineered allogeneic cells. Biomaterials. 2014; 35(2):779-791) showed that myogenic cells encapsulated in a biomimetic PEG-based hydrogel matrix could survive at high density for several months. In addition, a rapamycin-containing PEG coating has been shown to be able to improve the biocompatibility of alginate microcapsules during xenotransplantation (Park H S, Kim J W, Lee S H, et al. Antifibrotic effect of rapamycin containing polyethylene glycol-coated alginate microcapsule in islet xenotransplantation. J Tissue Eng Regen Med. 2017 11(4):1274-1284).

To improve encapsulated cell migration, attachment, proliferation and matrix remodeling, several different approaches have been explored. These include chemical modification of encapsulation materials by cross-linking with Arg-Gly-Asp (RGD; a cell adhesion motif) or gelatin (Sarker B, Rompf J, Silva R, et al. Alginate-based hydrogels with improved adhesive properties for cell encapsulation. Int J Biol Macromol. 2015; 78:72-78), as well as cell encapsulation in core-shell structured capsules (Agarwal P, Zhao S, Bielecki P, et al. One-step microfluidic generation of pre-hatching embryo-like core-shell microcapsules for miniaturized 3D culture of pluripotent stem cells. Lab Chip. 2013; 13(23):4525-4533; and Zhao S, Agarwal P, Rao W, et al. Coaxial electrospray of liquid core-hydrogel shell microcapsules for encapsulation and miniaturized 3D culture of pluripotent stem cells. Integr Biol (Camb). 2014; 6(9):874-884). As an example, multiple types of cells encapsulated within RGD peptide-modified alginate microcapsules displayed improved cell adhesion and proliferation (Dumbleton J, Agarwal P, Huang H, et al. The effect of RGD peptide on 2D and miniaturized 3D culture of HEPM cells, MSCs, and ADSCs with alginate hydrogel. Cell Mol Bioeng. 2016 June; 9(2): 277-288.). To generate a liquid core, alginate hydrogel beads may be coated with poly-L-lysine or chitosan before liquefying the center (Zhang W, Zhao S, Rao W, et al. A novel core-shell microcapsule for encapsulation and 3d culture of embryonic stem cells. J Mater Chem B Mater Biol Med. 2013; 2013(7):1002-1009). One-step fabrication of alginate core-shell microcapsules has been used to encapsulate embryonic stem cells with improved cell proliferation, aggregation and directed differentiation efficiency (Agarwal P, Zhao S, Bielecki P, et al. One-step microfluidic generation of pre-hatching embryo-like core-shell microcapsules for miniaturized 3D culture of pluripotent stem cells. Lab Chip. 2013; 13(23):4525-4533; and Zhao S, Agarwal P, Rao W, et al. Coaxial electrospray of liquid core-hydrogel shell microcapsules for encapsulation and miniaturized 3D culture of pluripotent stem cells. Integr Biol (Camb). 2014; 6(9):874-884).

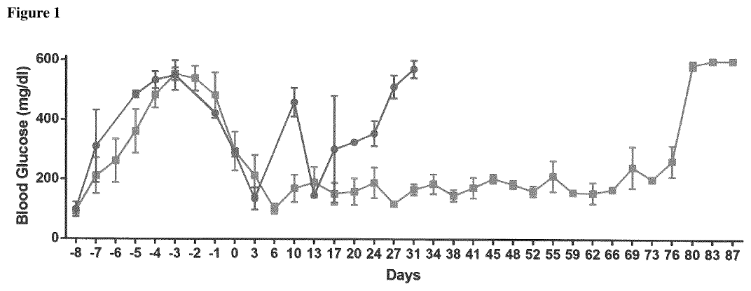

Alginate microcapsules may be used for islet transplantation in the clinical trials and several research efforts to protect isolated cells from immunological destruction. One of the challenges for long term function of these implants to treat patients with Type 1 diabetes is the immunogenicity of alginate microcapsules, where immune cells attack around the microcapsules and block it (a process known as fibrosis). We encapsulated exosomes inside the alginate microcapsules and found out that the immune response against these implants are significantly lower than the normal alginates. We therefore implanted pancreatic islets derived from rats, and encapsulated in our newly developed microcapsules. Then we made fully immunocompetent mice diabetic by injecting STZ, a common reagent used to create a mouse model for Type 1 diabetes. We found that while commercially available microcapsules could treat mice diabetics for 2-3 weeks, our developed microcapsules treat their diabetes for at least three months, and when grafts were removed, mice were again diabetic. This graft removal is an essential component to prove that the treatment of diabetes was through the transplants. We, as well as other researchers, have previously observed the myriad of immune activation following alginate capsule implantation, with macrophages being in the forefront of such immune responses (Vieseh, O. et al. 2015 Nature Materials 14: 643; Doloff, J. C. et al. 2017 Nature Materials 16: 671; and Rezaa, M. M. et al. 2018 Materials Today: Proceeding 5(7, part 3): 15580-15585). We also recently have observed the anti-inflammatory and immunomodulatory interaction of exomes from Mesenchymal Stem Cells (MSCs) with macrophages and T cells (Tsukamoto, K. et al. 2002 Journal of Atherosclerosis and Thrombosis 9(1): 57-64). We therefore hypothesized that incorporation of exosomes within the alginate microcapsules could reduce the immune response against alginates, leading to long term glycemic control of diabetic mice. FIG. 1 shows that alginate microcapsules incorporated with exosomes and pancreatic rat islets delays the graft rejection and extend the euglycemia of the diabetic mice for 3 months.

Exosome particle size in the disclosed hybrid microcapsule can be varied, for example from 10-500 nm in diameter, including 10 nm, 20 nm, 30 nm, 40 nm, 50 nm, 60 nm, 70 nm, 80 nm, 90 nm, 100 nm, 120 nm, 140 nm, 160 nm, 180 nm, 200 nm, 220 nm, 240 nm, 260 nm, 280 nm, 300 nm, 320 nm, 340 nm, 360 nm, 380 nm, 400 nm, 420 nm, 440 nm, 460 nm, 480 nm and 500 nm. Numbers of exosomes encapsulated in a hybrid microcapsule can range from 1×10⁵-1×10⁸, including 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, and 1×10⁸.

Alginate is a preferred biomaterial to encapsulate the islets for the long-term treatment of T1D. However, due to the endogenous fibrosis around the alginate capsules, the communication between islets and the in vivo niche gets disconnected. Our device, however, forms a protective biomaterial for islets prior to transplantation. This type of biomaterial not only could change the paradigm of islet transplantation, but also could influence the field of biomaterial-stem cell transplantation. Our current results have shown a 90 days delay in the graft rejection of transplanted islets as well as euglycemia.

A microcapsule is a small sphere with a uniform wall around it. The material inside the microcapsule is referred to as the core, internal phase, or fill, whereas the wall is sometimes called a shell, coating, or membrane. Some biocompatible materials like lipids and polymers, such as alginate, may be used as a mixture to trap the material of interest inside. Most microcapsules have pores with diameters between a few micrometers and a few millimeters. Exemplary coating materials are Ethyl cellulose, Polyvinyl alcohol, Gelatin and alginate, e.g., Sodium alginate.

Cells that have the capacity to release soluble factors such as cytokines, chemokines, insulin and growth factors which act in a paracrine or endocrine manner. These factors may act systemically or they may facilitate self-healing of an organ or region by inducing local (stem) cells or attracting cells to migrate towards the transplantation site. Such cells include cells that naturally secrete the relevant therapeutic factors, or which undergo epigenetic changes or genetic engineering that causes the cells to release large quantities of a specific molecular agent. Examples include cells that secrete factors that facilitate angiogenesis, anti-inflammation, glucose uptake and anti-apoptosis.

### Example 1

### Islet Xenotransplantation in Immunocompetent Diabetic Mice Using Stem Cell Derived Immunomodulatory Biomaterials

Foreign body response (FBR) to biomaterials compromises the function of implants and leads to medical complications. Here, we report a hybrid alginate microcapsule (AlgXO) that attenuated the immune response after implantation, through releasing exosomes derived from human Umbilical Cord Mesenchymal Stem Cells (XOs). Upon release, XOs suppress the local immune microenvironment, where xenotransplantation of rat islets encapsulated in AlgXO led to >170 days euglycemia in immunocompetent mouse model of Type 1 Diabetes. In vitro analyses revealed that XOs suppressed the proliferation of CD3/CD28 activated splenocytes and CD3+ T cells. Comparing suppressive potency of XOs in purified CD3+ T cells versus splenocytes, we found XOs more profoundly suppressed T cells in the splenocytes co-culture, where a heterogenous cell population is present. XOs also suppressed CD3/CD28 activated human peripheral blood mononuclear cells (PBMCs) and reduced their cytokine secretion including IL-2, IL-6, IL-12p70, IL-22, and TNFα. We further demonstrate that XOs mechanism of action is likely mediated via myeloid cells and XOs suppress both murine and human macrophages partly by interfering with NFκB pathway. We propose that through controlled release of XOs, AlgXO provide a promising new platform that could alleviate the local immune response to implantable biomaterials.

We hypothesized that co-transplantation of XOs within alginate microcapsules (AlgXO) would alleviate the FBR upon implantation. Upon blocking this inflammation, we next hypothesized that transplantation of rat islets within AlgXO would prolong the function of transplanted islets in immunocompetent streptozotocin-induced diabetic mice.

### Results

Islet Xenotransplantation within AlgXO Microcapsules Delays the Graft Rejection

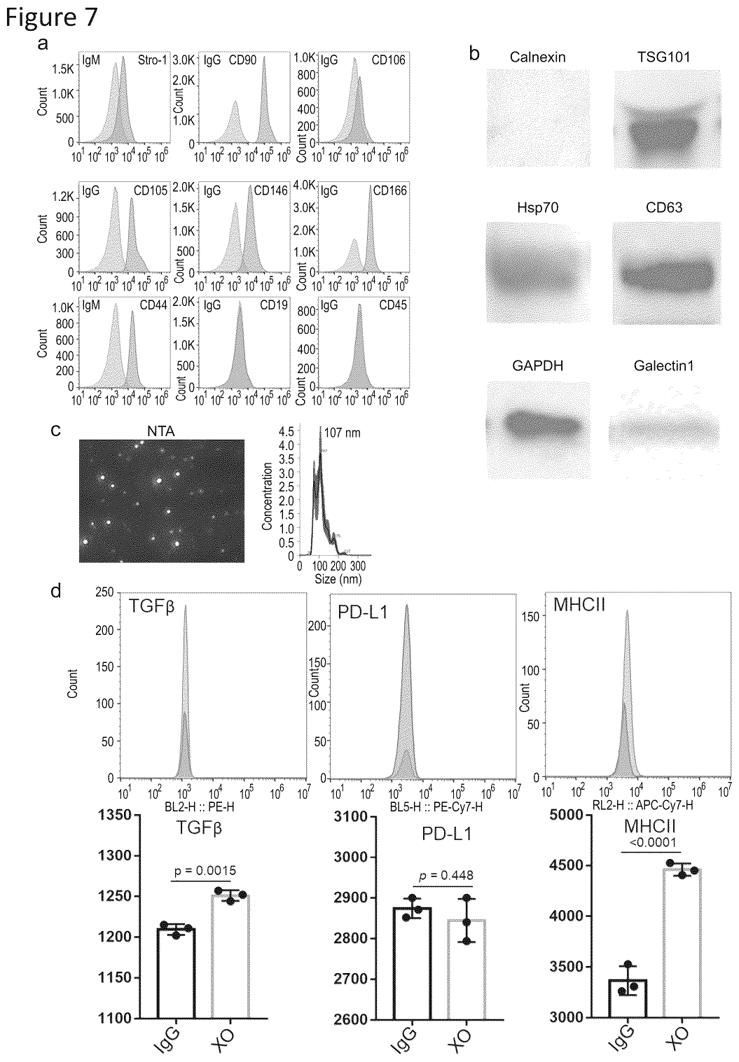

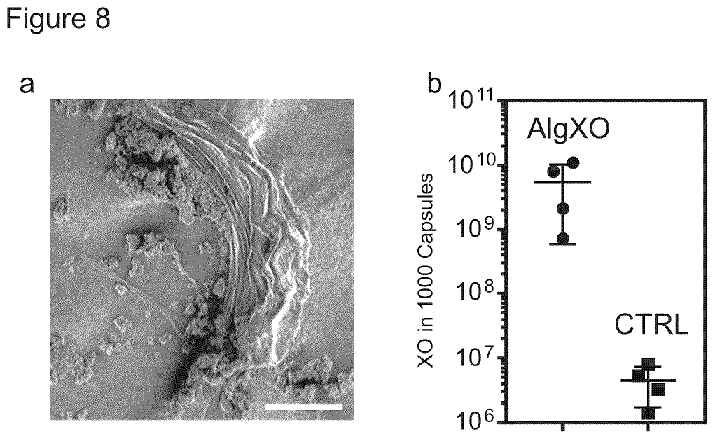

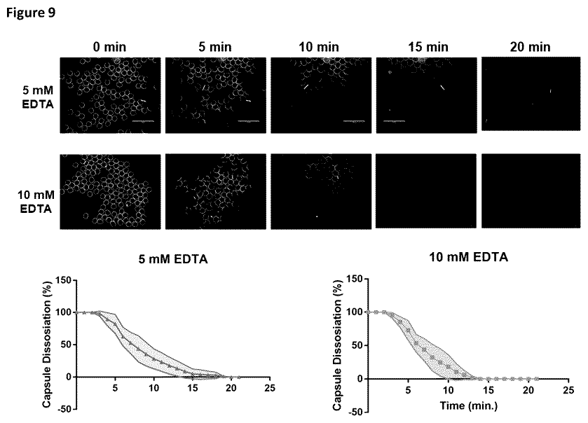

We first isolated XOs from umbilical cord-derived MSCs (UC-MSCs) and characterized UC-MSCs and the size, number, and protein biomarkers of their XOs (FIG. 7). We chose UC-MSCs due to their availability, noninvasive isolation, rapid proliferation, suitability for scale-up, and superior biological activity (Hass, R., Kasper, C., Bohm, S. & Jacobs, R. Different populations and sources of human mesenchymal stem cells (MSC): a comparison of adult and neonatal tissue-derived MSC. Cell Commun. Signal 9, 12-12 (2011)). Two types of alginate microcapsules were fabricated, which are regular Ba2+ cross-linked ultrapure alginate microcapsules (CTRL) and AlgXO. To fabricate AlgXO, we loaded XOs inside alginate microcapsules (FIG. 8, a). To quantify XOs within AlgXO, we dissolved microcapsules and collected XOs through ultracentrifugation (FIG. 9). Total number of XOs within 1000 AlgXO was 5.43×10⁹±4.84×10⁹ (n=4), whereas XOs within CTRL microcapsules were below the detection limit of nanoparticle tracking analysis (NTA; FIG. 8, b).

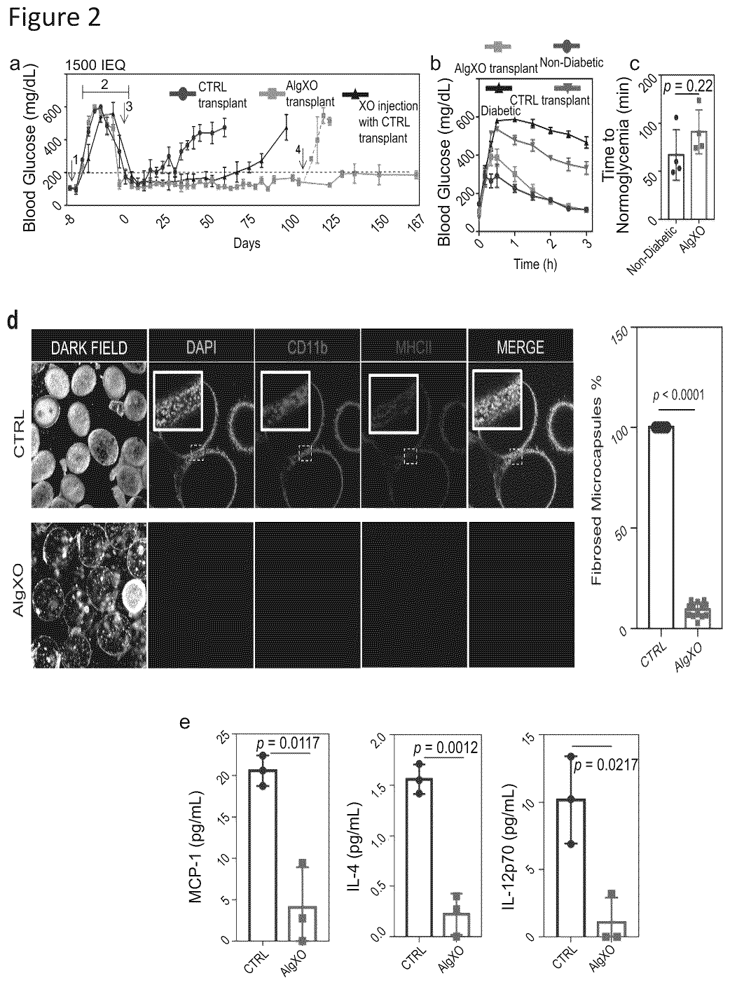

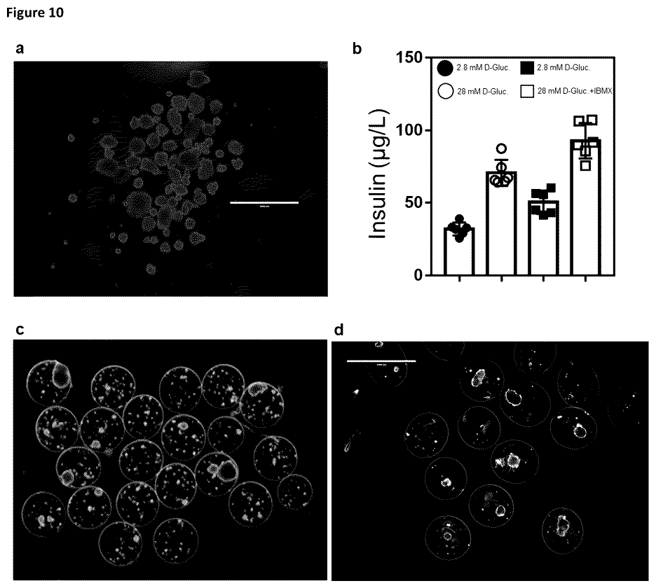

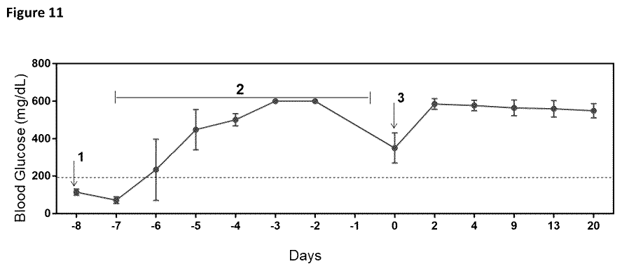

We then sought to investigate the functionality of islet transplantation within AlgXO microcapsules. Rat islets (1500 IEQ, islet equivalent) were encapsulated in either AlgXO or CTRL microcapsules and transplanted into the i.p. cavity of streptozotocin (STZ)-treated C57/BL6 mice with a week-long established hyperglycemia (n=5). To assure the purity and quality of rat islets from each isolation and minimize the batch-to-batch variations between islets, we conducted quality control for every batch (FIG. 10). FIG. 2, a shows that transplantation of rat islets within AlgXO provided euglycemia in diabetic mice for >170 days, whereas the islets transplanted within CTRL microcapsules functionally failed to regulate mice hyperglycemia within a month. To assure that the glycemic correction is due to AlgXO transplants and not beta cell regeneration in diabetic mice, we removed the AlgXO transplants after 105 days of transplantation by washing the i.p. cavity. This date was chosen because not only mice were normoglycemic, but the XO injected groups were hyperglycemic a month prior to it, allowing us to assure the graft function as well as superiority of AlgXO vs XO injected group. Within 16 h of graft removal, the non-fasting blood glucose was elevated and mice remained hyperglycemic (dashed line, FIG. 2, a). To control the effect of AlgXO on the maintenance of hyperglycemia in the STZ-induced diabetic mice, empty AlgXO microcapsules (i.e., without pancreatic islets) were also transplanted into the i.p. cavity of STZ-induced diabetic C57/BL6 mice, but they failed to reverse hyperglycemia (FIG. 11). We designed this experiment because a recent study has reported that intravenous injection of UC-MSCs derived XOs into STZ-induced diabetic mice promoted expression and membrane translocation of glucose transporter 4, and reduced the hyperglycemic severity (Sun, Y. et al. Human mesenchymal stem cell derived exosomes alleviate type 2 diabetes mellitus by reversing peripheral insulin resistance and relieving β-cell destruction. ACS Nano 12, 7613-7628 (2018)).

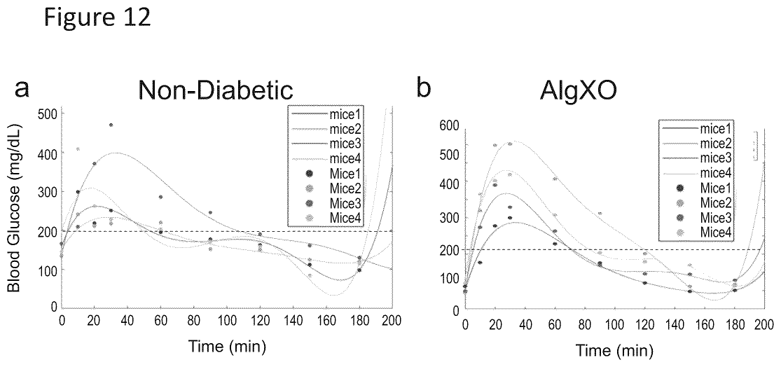

We next asked whether the in vivo function of islet xenotransplants could be prolonged by administration of nonencapsulated XOs. We thus transplanted 1500 IEQ rat islets in CTRL microcapsules, and at the same time, injected (i.p.) 8.1×10⁹±7.3×10⁸ XOs (n=4). This dose was chosen to be consistent with the dose of XOs in AlgXO xenotransplant studies conducted earlier. Administration of non-encapsulated XOs at the time of islet transplantation in CTRL microcapsules extended the euglycemia in diabetic mice for 2 months (FIG. 2, a), although not as prolonged as AlgXO transplants. We further tested the efficacy of AlgXO transplants in response to oral glucose tolerance test (OGTT) after 1 month of transplantation (FIG. 2, b). After 30 min of glucose challenge onset, the non-diabetic mice blood glucose reached to 291±120 mg/dL (n=4). During the same time, the blood glucose of mice transplanted with islets in AlgXO and CTRL microcapsules were 386±91 mg/dL and 534±9 mg/dL, respectively (n=4). This number was 580±28 mg/dL for diabetic mice (n=3). We set the 200 mg/dL as the normoglycemia threshold between diabetic (hyperglycemic) and non-diabetic (normoglycemic) mice. We then attempted to find the duration required for each mouse to reach normoglycemia after the glucose challenge. Polynomials with degree 5 were assigned to the OGTT curves (FIGS. 12, a and b), and time to normoglycemia was calculated based on the value of 200 for the polynomial functions. FIG. 2, c demonstrates that 65±27 minutes is the average required time for non-diabetic mice to reach normoglycemia after an OGTT, and such duration was 103±32 min for mice with AlgXO transplants. AlgXO transplants had a slightly higher time to reach normoglycemia after an OGTT compared to non-diabetic controls on average (n=6, p=0.063). This delay is likely due to the barrier of alginate network to the diffusion of insulin and glucose. These results suggest that AlgXO transplants had a comparable glucose response to non-diabetic mice during an OGTT, while none of the CTRL transplants achieved normoglycemia.

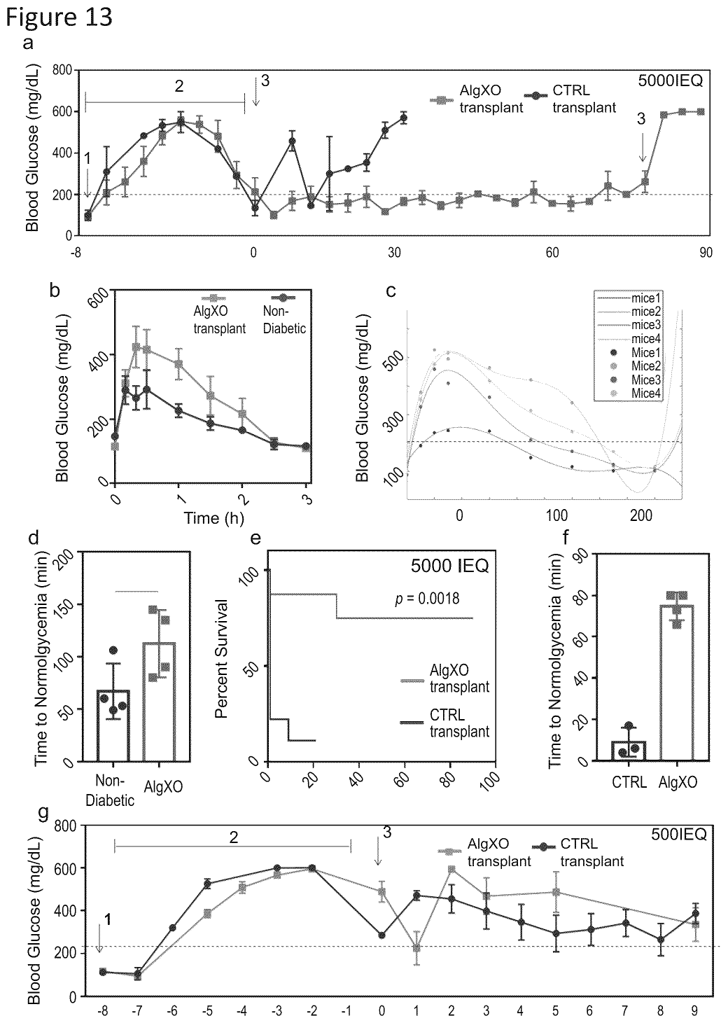

Clinical trials for islet transplantation have shown that allogeneic or xenogeneic source of islets affect the clinical efficacy and have led to conflicting results. Although xenotransplantation in non-immunosuppressed diabetic patients partially reduced hypoglycemic events, higher doses of xenogeneic islets were less effective (Matsumoto, S. et al. Clinical porcine islet xenotransplantation under comprehensive regulation. Transplant. Proc. 46, 1992-1995 (2014); and Ekser, B., Bottino, R. & Cooper, D. K. C. Clinical islet xenotransplantation: a step forward. EBioMedicine 12, 22-23 (2016)). We further performed AlgXO-encapsulated islet dose study to establish a therapeutic dose (FIG. 13, Islet Dose Study), where we found that 1500 IEQ rat islets showed longer euglycemic induction than 5000 IEQ, but a lower 500 IEQ failed to correct mice hyperglycemia.

### AlgXO Reduces Inflammation and Fibrosis

We next sought to delineate possible mechanisms that prolonged the function of islet transplants within AlgXO microcapsules. In a broad context, two major players are widely recognized in the long-term failure of microencapsulation technologies: (1) lack of nutrients and oxygen accessibility into the microcapsules, which leads to islet necrosis (Evron, Y. et al. Long-term viability and function of transplanted islets macroencapsulated at high density are achieved by enhanced oxygen supply. Sci. Rep. 8, 6508 (2018)), and (2) and inflammatory-based foreign body response (FBR) within weeks of transplantation forms a dense fibrotic tissue around the microcapsules, blocking the function of islets (Doloff, J. C. et al. Colony stimulating factor-1 receptor is a central component of the foreign body response to biomaterial implants in rodents and nonhuman primates. Nat. Mater. 16, 671 (2017); Bochenek, M. A. et al. Alginate encapsulation as long-term immune protection of allogeneic pancreatic islet cells transplanted into the omental bursa of macaques. Nat. Biomed. Eng. 2, 810-821 (2018); and Rezaa Mohammadi, M., Rodrigez, S., Cao, R., Alexander, M. & Lakey, J. R. T. Immune response to subcutaneous implants of alginate microcapsules. Mater. Today.: Proc. 5, 15580-15585 (2018)). To investigate possible mechanisms by which islets encapsulated within AlgXO provide longer glycemic correction, we examined both of these points (Bochenek, M. A. et al. Alginate encapsulation as long-term immune protection of allogeneic pancreatic islet cells transplanted into the omental bursa of macaques. Nat. Biomed. Eng. 2, 810-821 (2018); Farah, S. et al. Long-term implant fibrosis prevention in rodents and nonhuman primates using crystallized drug formulations. Nat. Mater. 18, 892-904 (2019); de Vos, P., Hamel, A. F. & Tatarkiewicz, K. Considerations for successful transplantation of encapsulated pancreatic islets. Diabetologia 45, 159-173 (2002) and Vaithilingam, V. & Tuch, B. E. Islet transplantation and encapsulation: an update on recent developments. Rev. Diabet. Stud. 8, 51 (2011)).

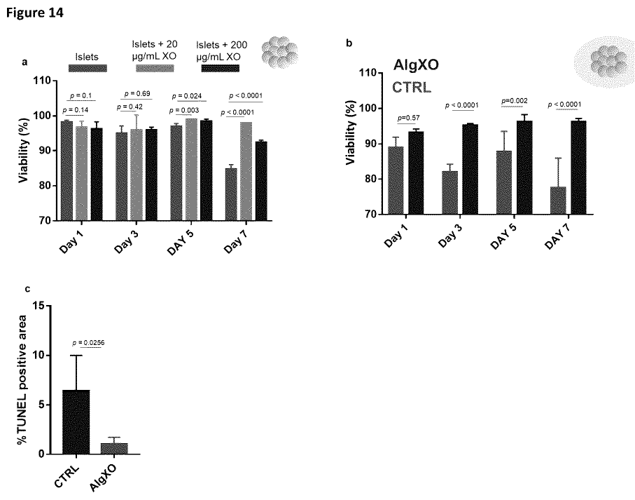

In the early stage of transplantation, the health and viability of islets suffer from oxidative stress (likely within a week), and at later stages inflammatory-induced fibrosis influences graft viability by constraining oxygen and metabolite diffusion into the microcapsules (Bochenek, M. A. et al. Alginate encapsulation as long-term immune protection of allogeneic pancreatic islet cells transplanted into the omental bursa of macaques. Nat. Biomed. Eng. 2, 810-821 (2018)). Recently, it has been demonstrated that MSC exosomes could relieve the β-cell apoptosis and destruction (Sun, Y. et al. Human mesenchymal stem cell derived exosomes alleviate type 2 diabetes mellitus by reversing peripheral insulin resistance and relieving β-cell destruction. ACS Nano 12, 7613-7628 (2018)), and enhance islets survival under hypoxic conditions (Nie, W. et al. Human mesenchymal-stem-cells-derived exosomes are important in enhancing porcine islet resistance to hypoxia. Xenotransplantation 25, e12405 (2018)). Thus, we speculated that XOs may enhance rat islets viability in vitro and found that XOs (both 20 and 200 μg/mL doses) as well as AlgXO enhance the rat islets viability (FIGS. 14, a and b). It is therefore likely that AlgXO retains the encapsulated islets viability during early stages of transplantation. For longer time periods after transplantation, we sought to understand the viability of islets. Microcapsules from both groups were explanted 1 month after implantation and TUNEL assay was conducted to compare the viability of islets. FIG. 14, c shows the results of TUNEL assay, where after 1 month of transplantation, the TUNEL positive area of islets transplanted within AlgXO (1.02%±0.32%) was higher (n=5, p=0.0256) compared to CTRL (6.44%±1.59%). This suggests that after 1 month of transplantation, islets within AlgXO possess higher viabilities.

In later stages of transplantation, however, inflammatory-led FBR further compromise the viability and functionality of the islets within microcapsules. Inhibition of inflammation-led fibrosis has been shown to improve long-term function of transplanted islets and euglycemia in diabetic rodents (Vegas, A. J. et al. Long-term glycemic control using polymer-encapsulated human stem cell-derived beta cells in immune-competent mice. Nat. Med. 22, 306-311 (2016); Bochenek, M. A. et al. Alginate encapsulation as long-term immune protection of allogeneic pancreatic islet cells transplanted into the omental bursa of macaques. Nat. Biomed. Eng. 2, 810-821 (2018); Farah, S. et al. Long-term implant fibrosis prevention in rodents and nonhuman primates using crystallized drug formulations. Nat. Mater. 18, 892-904 (2019); and Vegas, A. J. et al. Combinatorial hydrogel library enables identification of materials that mitigate the foreign body response in primates. Nat. Biotechnol. 34, 345 (2016)). Recognizing the multi-potent anti-inflammatory properties of MSCs derived XOs (Riazifar, M. et al. Stem cell-derived exosomes as nanotherapeutics for autoimmune and neurodegenerative disorders. ACS Nano 13, 6670-6688 (2019); Zhang, B. et al. Mesenchymal stromal cell exosome-enhanced regulatory Tcell production through an antigen-presenting cell-mediated pathway. Cytotherapy 20, 687-696 (2018); and Bai, L. et al. Effects of mesenchymal stem cell-derived exosomes on experimental autoimmune uveitis. Sci. Rep. 7, 4323 (2017)), we hypothesized that encapsulation of rat islets within AlgXO microcapsules reduces the inflammatory response, leading to the long-term function of islets and glycemic control in immunocompetent diabetic mice. To investigate the inflammatory response against AlgXO and CTRL xenotransplants, we explanted both groups and analyzed for immune infiltration

Since the 1500 IEQ CTRL transplants failed to function within about 1 month (FIG. 2, a), we explanted CTRL and AlgXO xenotransplants from mice at day 31 of the implantation. We next analyzed the pericapsular attachment around both microcapsules and observed that CTRL groups are covered with CD11b+ cells, while most of AlgXO explants were clear and transparent (FIG. 2, d). It is noteworthy that 9%±3.6% of the microcapsules from AlgXO explants showed pericapsular cell attachment that was significantly lower than pericapsular cell attachment on CTRL explants (FIG. 2, d, p<0.0001). Analyses of the subtypes that infiltrated around CTRL microcapsules revealed the presence of CD11b+ myeloid-derived cells. At least in some locations, CD11b+ cells express MHCII+, as observed through co-localization of CD11b and MHCII markers. As one of the main myeloid-derived cells, macrophages generally express moderate levels of MHCII to regulate immune tolerance and local surveillance to maintain homeostatic immunity. However, macrophages will upregulate MHCII expression and antigen presentation capacity in a proinflammatory environment, where antigens can be presented to CD4+ lymphocytes.

To better understand the effect of XOs on the pericapsular environment, we explanted the grafts 1 month after transplantation and analyzed the lavage solution obtained from the explants. Analyses of cytokines and chemokines demonstrated reduced secretion of MCP-1 (20.6±1.8 pg/ml to 4.1±4.96 pg/ml, n=3, p=0.0117), IL-4 (1.6±0.2 pg/ml to 0.2±0.2 pg/ml, n=3, p=0.0012), and IL-12p70 (6.7±6.6 pg/ml to 1.1±1.8 pg/ml, n=3, p=0.0217) in the pericapsular area of AlgXO transplants compared to controls (FIG. 2, e). MCP-1 mediates the recruitment of inflammatory monocytes to the site of inflammation and IL-12p70 These results in their totality suggest less recruitment of immune cells to the AlgXO compared to CTRL microcapsules.

Next, we sought to further understand the mechanisms for the observed differential inflammatory responses against AlgXO and CTRL transplants. Alginate immunogenicity has been attributed to two separate mechanisms, which could also be viewed as complementary phenomena. First, prior studies have shown endotoxin contaminations within alginate are the main immunogens, including lipopolysaccharide (LPS), lipoteichoic acid, and peptidoglycans (Paredes-Juarez, G. A., de Haan, B. J., Faas, M. M. & de Vos, P. The role of pathogen-associated molecular patterns in inflammatory responses against alginate-based microcapsules. J. Control. Release 172, 983-992 (2013); and Paredes Juarez, G. A., Spasojevic, M., Faas, M. M. & de Vos, P. Immunological and technical considerations in application of alginate-based microencapsulation systems. Front. Bioeng. Biotechnol. 2, 26 (2014)). Through lack of endotoxin presence within the commercially purified alginate (such as the UPLVG in the present study), others have reported that even without endotoxins alginate may enhance immune response (Doloff, J. C. et al. Colony stimulating factor-1 receptor is a central component of the foreign body response to biomaterial implants in rodents and nonhuman primates. Nat. Mater. 16, 671 (2017); and Vegas, A. J. et al. Combinatorial hydrogel library enables identification of materials that mitigate the foreign body response in primates. Nat. Biotechnol. 34, 345 (2016)). We recently have shown that even an ultrapure alginate could stimulate macrophages to an inflammatory lineage (Mohammadi, M. et al. Controlled release of stem cell secretome attenuates inflammatory response against implanted biomaterials. Adv. Healthc. Mater. 9, e1901874 (2020)). These reports suggest that such inflammatory response is likely due to the inherent nature of alginate. Guluronate oligosaccharide derived from alginate, for example, has been reported to readily activate macrophages partly through Toll-like receptor 4 (TLR4) signaling pathway (Mohammadi, M. et al. Controlled release of stem cell secretome attenuates inflammatory response against implanted biomaterials. Adv. Healthc. Mater. 9, e1901874 (2020); and Fang, W. et al. Identification and activation of TLR4-mediated signalling pathways by alginate-derived guluronate oligosaccharide in RAW264.7 macrophages. Sci. Rep. 7, 1663 (2017)). While the exact mechanisms for such response is debated, resolving the inflammatory response against alginate microcapsules is unanimously reported to prevent or delay the fibrosis. In this context, many groups have reported the long-term efficacy of islet transplantation within fibrosis-resistant devices (Farah, S. et al. Long-term implant fibrosis prevention in rodents and nonhuman primates using crystallized drug formulations. Nat. Mater. 18, 892-904 (2019); Vegas, A. J. et al. Combinatorial hydrogel library enables identification of materials that mitigate the foreign body response in primates. Nat. Biotechnol. 34, 345 (2016); Alagpulinsa, D. A. et al. Alginate-microencapsulation of human stem cell-derived 13 cells with CXCL12 prolongs their survival and function in immunocompetent mice without systemic immunosuppression. Am. J. Transplant. 19, 1930-1940 (2019); and Liu, Q. et al. Zwitterionically modified alginates mitigate cellular overgrowth for cell encapsulation. Nat. Commun. 10, 5262 (2019)).

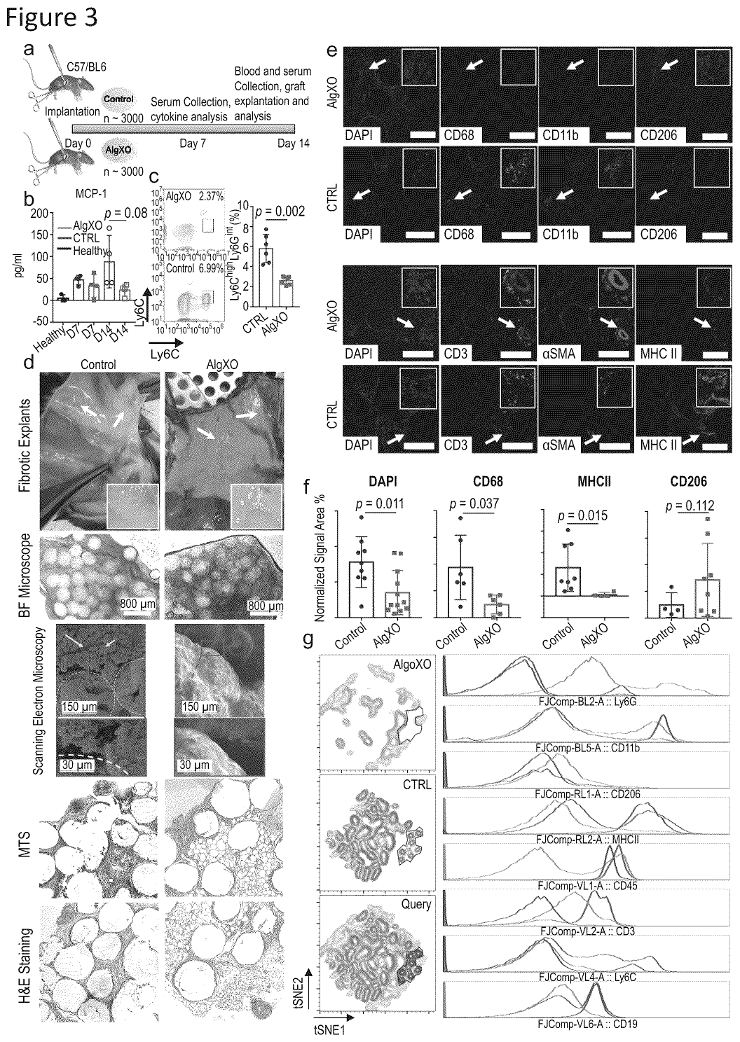

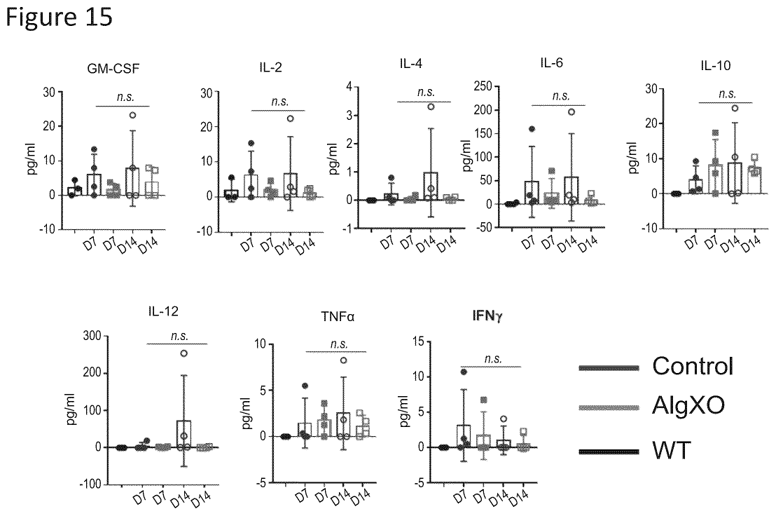

To comprehensively compare the inflammatory response of AlgXO and CTRL microcapsules, we further focused on the empty microcapsules and the inflammatory response they induce in vivo. We transplanted 3000 AlgXO or CTRL microcapsules into the subcutaneous space of C57/BL6 mice, and both microcapsules were explanted after 2 weeks (FIG. 3, a). A 2 weeks timepoint was selected, since it has been established as a suitable timepoint to resolve and reflect both innate and adaptive immune system as well as fibrotic responses to implanted materials in C57/BL6 mice16,64. At days 7 and 14 post-transplant serum cytokines were also measured, reflecting the systemic inflammatory response, if any. Among 11 cytokines examined, there was no significant difference between serum cytokines of mice that were subcutaneously transplanted with AlgXO or CTRL (FIG. 3, b and FIG. 15). While not statistically significant, the average amount of MCP-1 chemokine in the serum of mice transplanted with CTRL microcapsules for 2 weeks (87.8±59.9 pg/mL) was 3.7-fold higher than mice implanted with AlgXO microcapsules (23.3±13.4 pg/mL). The difference between systemic MCP-1 led us to further study the circulatory inflammatory monocytes in response to AlgXO and CTRL transplants.

Detection of circulating microbial molecules or proinflammatory cytokines by bone marrow-resident cells leads to MCP-1 production to modulate the frequency of circulating inflammatory monocytes65. MCP-1 is a chemokine that binds to CCR2 and mediates the recruitment of inflammatory (Ly6Chigh) monocytes to the site of inflammation. We next sought to quantify the inflammatory monocytes in the mice's blood 2 weeks after implantation. FIG. 3, c shows the flow cytometry plots and their quantification of the inflammatory monocytes (CD45+CD11b+Ly6ChighLy6Gmed)66 subpopulation. CD45+CD11b+Ly6ChighLy6Gmed in the mice transplanted with AlgXO (2.62%±0.4%) were significantly lower (n=3, p=0.002) compared to those monocytes in the blood circulation of CTRL microcapsules (5.8%±1.4%). These observations suggest that the AlgXO transplants are likely to reduce the systemic inflammatory response against alginate microcapsules.

The destination of circulating monocyte has been linked to the site of MCP-1 secretion (Lacey, D. C. et al. Defining GM-CSF- and macrophage-CSF-dependent macrophage responses by in vitro models. J. Immunol. 188, 5752 (2012); and Yoshimura, T. The chemokine MCP-1 (CCL2) in the host interaction with cancer: a foe or ally? Cell. Mol. Immunol. 15, 335-345 (2018)), which is also a site of hyperinflammation. In the present study, the transplant site is likely to be the main site of inflammatory response (Mohammadi, M. et al. Controlled release of stem cell secretome attenuates inflammatory response against implanted biomaterials. Adv. Healthc. Mater. 9, e1901874 (2020)). We therefore sought to investigate the local inflammatory response around the implants 2 weeks after transplantation. We found that all detectable CTRL microcapsules had agglomerated into clumps of alginate aggregates (FIG. 3, d). For AlgXO, while some microcapsules were remained intact and non-aggregated (shown with white arrows), the rest were entrapped in a pseudo tissue with multiple blood vessels around them. These tissues from both groups were isolated carefully so as not to contain endogenous tissues of mice. Bright Field microscopy demonstrated that pseudo tissues have entrapped microcapsules (FIG. 3, d). Under scanning electron microscopy evaluations, some microcapsules were detected (FIG. 3, d; white dashed line for visual guide of microcapsules) were surrounded with rough microstructures and the AlgXO ones were entrapped in smooth structures. Tissues were sectioned into 5-10-μ slices and stained with H&E and Masson's trichrome staining (MTS). The fibrotic tissue formed around CTRL microcapsules demonstrated the significant infiltration of mostly mononuclear cells, while such histology was not observed in AlgXO fibrotic tissues (FIG. 3, d).

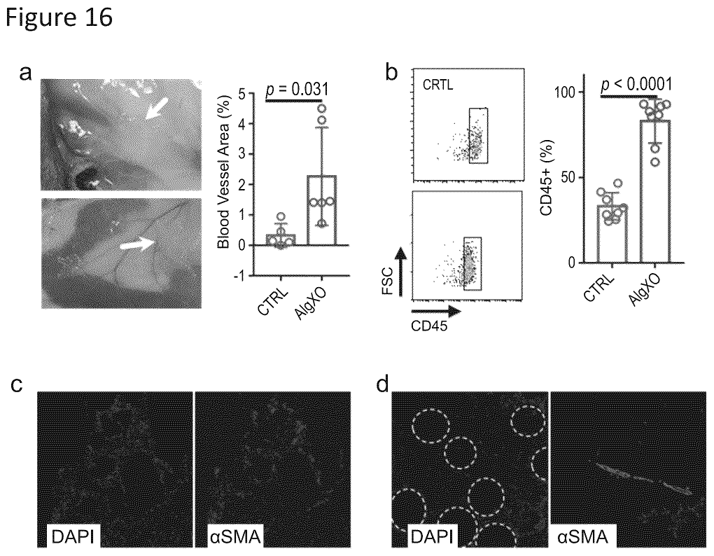

To better compare the immune environment, we compared and quantified the cellular components that drive the fibrotic response. Both fibrotic tissues were stained for different immunocytes including macrophages (CD11b+ and CD68+), T cells (CD3+), pro-regenerative macrophages (CD206), antigen-presenting cells (MHCII), and fibrotic marker of smooth muscle actin (αSMA). DAPI counterstaining was also used to count total cell infiltration within fibrotic tissues (FIG. 3, e, and FIG. 16). FIG. 3, f shows that total cell infiltration around microcapsules was significantly lower in AlgXO fibrotic tissues (p=0.011). Similar trends were observed for CD68 (p=0.037) and MHCII (p=0.015). In contrast, there was no association between CD206 expression (p=0.112).

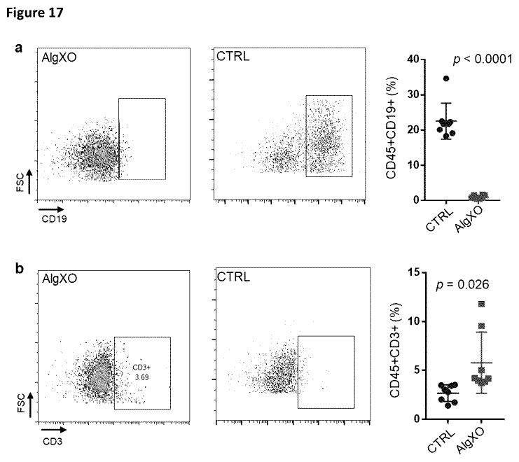

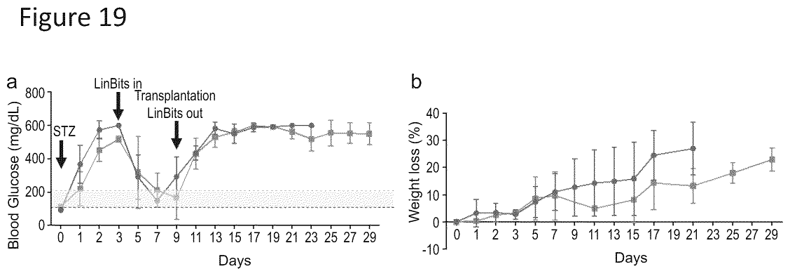

To gain more holistic information on the fibrotic tissues around CTRL and AlgXO microcapsules, we compared the components of both microcapsules at the cellular level using flow cytometry. The tSNE plots in FIG. 3, g demonstrate highly segregated subpopulations for AlgXO and CTRL fibrotic tissues. We particularly queried the subpopulations that were absent in AlgXO but present in CTRL as shown in FIG. 3, g black line area (query). This subpopulation is CD45+CD11b+CD19+MHCII+CD3−Ly6C−, which is likely to be the memory B cells subpopulation. To further assess the quantity of B cells in fibrotic microenvironments, we analyzed CD45+CD19+ B cells (FIG. 17, a). The CD45+CD19+ cells were remarkably fewer (n=4, p<0.0001) in AlgXO (0.9%±0.5%) compared to that of CTRL (22.5%±5.1%). B lymphocytes play critical roles in the FBR against alginate microcapsules. In particular, genetic deletion of B cells as well as CXCL13 neutralization have been reported to dampen the FBR to implanted alginate microcapsules during a 2-weeks implantation period (Doloff, J. C. et al. Colony stimulating factor-1 receptor is a central component of the foreign body response to biomaterial implants in rodents and nonhuman primates. Nat. Mater. 16, 671 (2017)), which aligns with our observations in the present study. In addition to B cells, innate lymphoid cells and γδ+ T cells lead to a chronic adaptive antigen-dependent Th17 cell response (Chung, L. et al. Interleukin 17 and senescent cells regulate the foreign body response to synthetic material implants in mice and humans. Sci. Transl. Med. 12, eaax3799 (2020)). In our study, we found that there was a higher quantity of CD3+ in AlgXO compared to CTRL (p=0.026), which is likely due to the blood/blood vessels in the AlgXO microenvironment (FIG. 17, b). This could be further confirmed due to the vicinity of blood vessels with T cells (FIG. 3, e). While inflammatory response was reduced around subcutaneous AlgXO microcapsules, transplantation of 155 IEQ rat islets within the subcutaneous space of diabetic mice did not restore the euglycemia (FIG. 19), which is likely due to the observed fibrotic response.

### AlgXO's Reduced Foreign Body Response is Partly Due to the Releasing of Exosomes in a Controlled Fashion

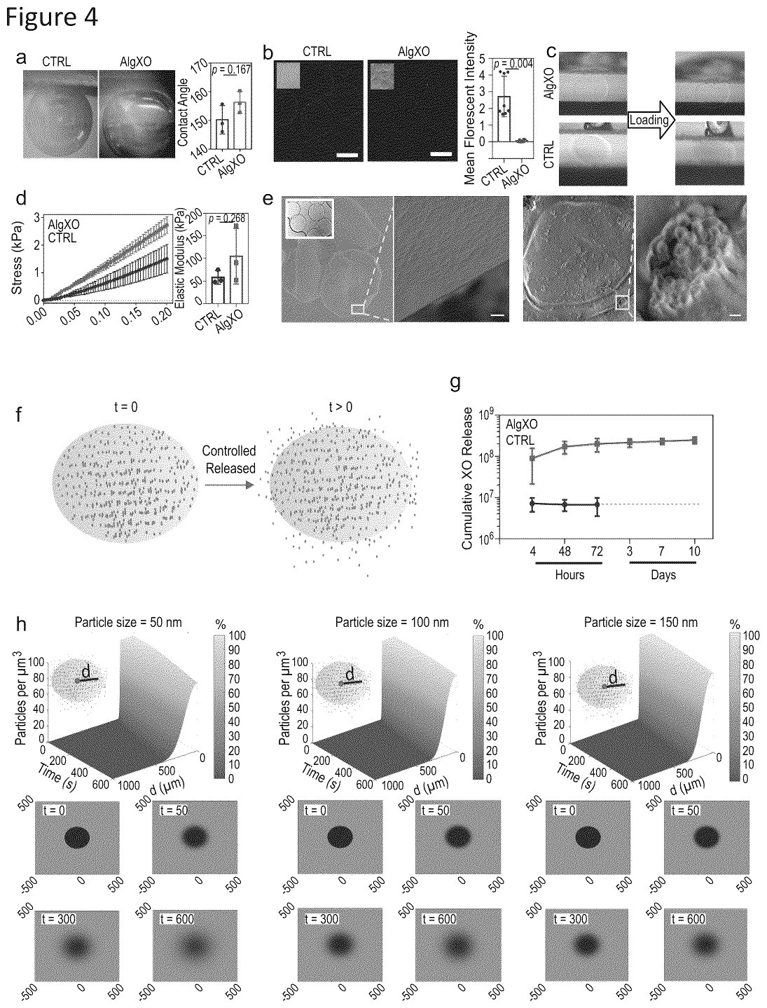

We pursued our investigation to delineate the effect of XOs controlled release on the AlgXO's reduced inflammatory response. We first characterized the physical and mechanical properties of AlgXO and compared them against CTRL microcapsules, as these properties remarkably influence the biological response of biomaterials. Water interactions with biomaterial surface, for example, have been recognized as a fundamental characteristic determining the immunological responses of biomaterials. Compared to hydrophilic materials, hydrophobic (and slightly hydrophilic) biomaterials adsorb more proteins. This is mainly because proteins adjacent to hydrophilic surfaces must displace more water molecules bound to biomaterial surface (Mohammadi, M. R., Luong, J. C., Kim, G. G., Lau, H. & Lakey, J. R. T. in Handbook of Tissue Engineering Scaffolds, Vol. 1 (eds Mozafari, M., Sefat, F. & Atala, A.) (Woodhead Publishing, 2019); and Seong, S.-Y. & Matzinger, P. Hydrophobicity: an ancient damage-associated molecular pattern that initiates innate immune responses. Nat. Rev. Immunol. 4, 469-478 (2004)). We thus sought to measure the contact angle for AlgXO and CTRL biomaterials using captive bubble contact angle method. FIG. 4, a shows that the contact angle for AlgXO (156.3°±3.8°) was higher than that of CTRL microcapsules (150.2°±4.9°). This suggests that AlgXO is slightly more hydrophobic; however, there was no significant correlation (n=3, p=0.167). Importance of hydrophobicity lies in protein adsorption onto the biomaterials surface, which has been linked with the FBR. Many studies have reported that the blocking of protein adsorption of biomaterials silences the immune response (Vegas, A. J. et al. Combinatorial hydrogel library enables identification of materials that mitigate the foreign body response in primates. Nat. Biotechnol. 34, 345 (2016); and Yesilyurt, V. et al. A facile and versatile method to endow biomaterial devices with zwitterionic surface coatings. Adv. Healthc. Mater. 6, 1601091 (2017)). These proteins may include components of the coagulation cascade (fibrinogen and tissue factors), complement cascade (C5), and other plasma-derived proteins (albumin and IgG) (Anderson, J. M., Rodriguez, A. & Chang, D. T. Foreign body reaction to biomaterials. Semin. Immunol. 20, 86-100 (2008)). IgG and fibronectin adsorption led the Mac-1-mediated attachment of neutrophils and macrophages to biomaterial surfaces during the acute phase of inflammation (Hu, W. J., Eaton, J. W. & Tang, L. Molecular basis of biomaterial-mediated foreign body reactions. Blood 98, 1231-1238 (2001)). We therefore attempted to investigate the IgG adsorption onto both AlgXO and CTRL microcapsules. IgG adhered to the surface of CTRL microcapsules more pronouncedly (2.70%±1.21%, n=3, p=0.0004) compared to AlgXO (0.05%±0.06%) (FIG. 4, b). The less-fibrotic properties AlgXO could partly originate from the less protein adsorption onto its surface. Proteins absorption onto the biomaterials and their conformation could lead to the formation of different biomaterial-associated molecular patterns, initiating the inflammatory response (Eslami-Kaliji, F., Sarafbidabad, M., Rajadas, J. & Mohammadi, M. R. Dendritic cells as targets for biomaterial-based immunomodulation. ACS Biomater. Sci. Eng. 6, 2726-2739 (2020)).

Mechanical properties of biomaterials have been linked to immunological response of implants. For instance, macrophage confinement reduces their inflammatory response through reduction in actin polymerization and LPS-stimulated nuclear translocation of MRTF-A (Jain, N. & Vogel, V. Spatial confinement downsizes the inflammatory response of macrophages. Nat. Mater. 17, 1134-1144 (2018)). In addition, macrophages adhere to stiff surfaces more profoundly (Meli, V. S. et al. Biophysical regulation of macrophages in health and disease. J. Leukoc. Biol. 106, 283-299 (2019)). We thus attempted to characterize the mechanical properties of both AlgXO and CTRL microcapsules (FIGS. 4, c and d). FIG. 4, c demonstrates the images of the initial and final vertical positions of the cantilevers, exerting pressure on the microcapsules. Stress-strain curves (FIG. 4, d) shows the linear behavior, where the difference between elastic modulus of AlgXO (104.7±61.4 kPa) and CTRL (57.8±14.9 kPa) was not significant (n=3, p=0.268).

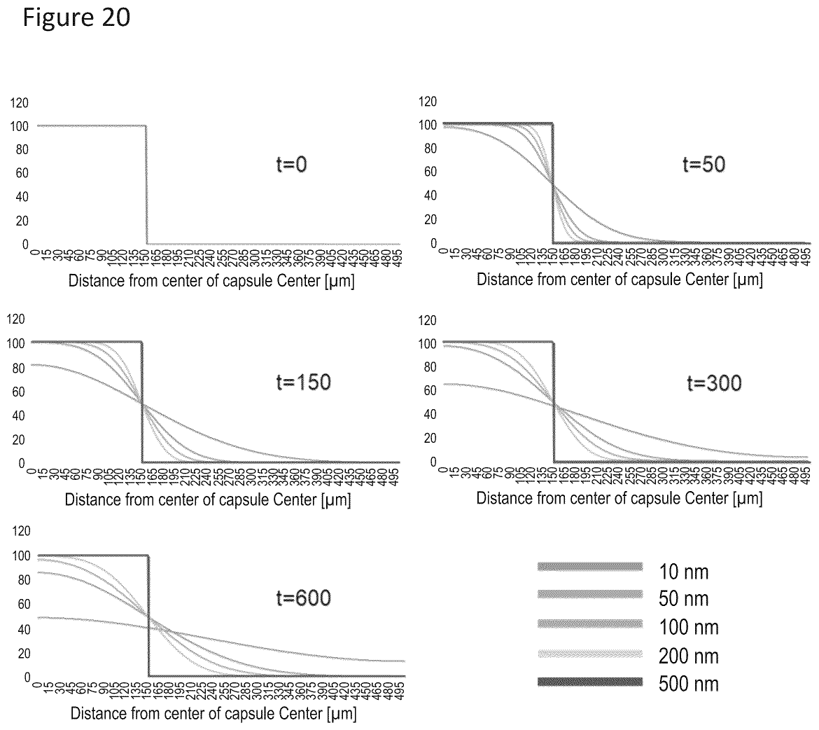

We further asked other possible mechanisms that are likely to play roles in AlgXO's immunomodulatory properties. Our initial hypothesis was that the immunomodulatory effects of AlgXO is partly due to the release of XOs. MSC-derived XOs have been demonstrated to possess immunosuppressive functions both in vitro (Pacienza, N. et al. In vitro macrophage assay predicts the in vivo anti-inflammatory potential of exosomes from human mesenchymal stromal cells. Mol. Ther. Methods Clin. Dev. 13, 67-76 (2019)) and in rodent models (Lankford, K. L. et al. Intravenously delivered mesenchymal stem cell-derived exosomes target M2-type macrophages in the injured spinal cord. PLoS ONE 13, e0190358 (2018)). To better understand this possibility, we sought to find whether XOs within AlgXO release into the surrounding microenvironment of microcapsules. We first visualized and compared the CTRL and AlgXO microcapsules using Scanning electron microscopy (SEM) on air-dried microcapsules. FIG. 4, e demonstrates SEM micrographs of both AlgXO and CTRL microcapsules, suggesting the presence of surface pores in the 50-200 nm size scale. SEM micrographs of AlgXO microcapsules demonstrated the encapsulation of spherical-shaped vesicles within AlgXO, and their possible release from the surface of a microcapsule (FIG. 4, e, right panel, scale=1 μm). We particularly hypothesized that XOs could be released from AlgXO (FIG. 4, f) as they readily diffuse within the nano-meshes of extracellular matrix and communicate over long distances within the body. Recently, it has been demonstrated that due to the aquaporin-1 mediated XOs deformability, XOs could transport within and diffuse outwards of alginate matrix (as well as extracellular matrices), despite XOs being larger than the mesh size of the surrounding network (Lenzini, S., Bargi, R., Chung, G. & Shin, J.-W. Matrix mechanics and water permeation regulate extracellular vesicle transport. Nat. Nanotechnol. 15, 217-223 (2020)). We next incubated AlgXO microcapsules in vitro and measured the release of XOs, demonstrating the controlled release of XOs over the course of 10 days (FIG. 4, g). We found that 3.03×10⁰⁷±2.75×10⁰⁷ XOs are released within 10 min of culturing, and total release increases to 2.49×10⁰⁸±4.63×10⁰⁷ XOs within 10 days. It should be noted that total XOs encapsulated is 5.43×10⁰⁹±4.84×10⁰⁹ (FIG. 8, b). To further understand the release profile of XOs, we modeled the release of exosomes based on the Fickian diffusion of nanoparticles ranging from 50 to 150 nm in diameter (i.e., the size range of XOs). FIG. 4, h demonstrates the simulated diffusion of XOs with 50, 100, and 150 nm. The top panels are time (s) vs. particles concentration (per μm³) vs. distance from capsules center (μm). Bottom panels on FIG. 4, h demonstrate the heatmap representation of the XOs diffusion outwards of microcapsules. In these maps the 1 mm×1 mm diffusion microenvironment is shown, and the blue color represents the diffusion. These simulations suggest that smaller particles (50 nm of diameter) diffuse faster than 150 nm particles. To check our simulation models beyond the scale of 50-150 nm, smaller (i.e., 10 nm) and larger (200 and 500 nm) particles were input into the code. It was found that in 600 s, while 10 nm possess expedited diffusion rates, 500 nm particles remain within the microcapsules and no outward diffusion was obtained (FIG. 20).

### XOs Suppress Murine Macrophages and T Lymphocytes

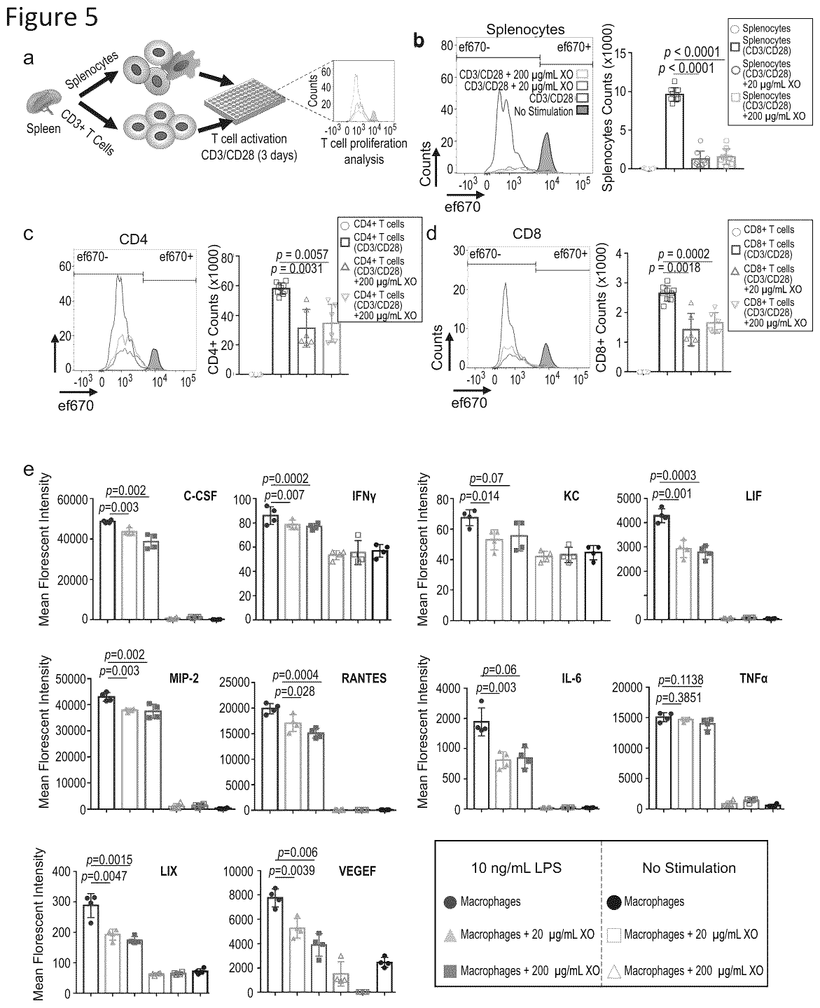

Similar to the suppressive properties of their parental cells, XOs derived from MSCs have been demonstrated to possess immunosuppressive functions both in vitro (Pacienza, N. et al. In vitro macrophage assay predicts the in vivo anti-inflammatory potential of exosomes from human mesenchymal stromal cells. Mol. Ther. Methods Clin. Dev. 13, 67-76 (2019)) and in rodent models (Lankford, K. L. et al. Intravenously delivered mesenchymal stem cell-derived exosomes target M2-type macrophages in the injured spinal cord. PLoS ONE 13, e0190358 (2018)). We recently showed that bone marrow-derived MSC XOs suppress human peripheral blood mononuclear cells (PBMCs) upon activation with anti-CD3/CD28 stimulation (Riazifar, M. et al. Stem cell-derived exosomes as nanotherapeutics for autoimmune and neurodegenerative disorders. ACS Nano 13, 6670-6688 (2019)). In the splenocyte cocultures supplemented with IL-2, bone marrow-derived MSC XOs also induced CD4+CD25+FoxP3+ regulatory T cells (Riazifar, M. et al. Stem cell-derived exosomes as nanotherapeutics for autoimmune and neurodegenerative disorders. ACS Nano 13, 6670-6688 (2019)). To understand the mechanisms by which XOs (derived from umbilical cords) exert their suppressive function, here we first studied their effects on activated murine splenocyte and then on purified CD3+ T cells isolated from splenocytes (FIG. 5, a). Cell Proliferation Dye eFluor670-labeled splenocytes from C57/BL6 wild-type mice were stimulated with plate-bound anti-CD3 and anti-CD28 in vitro in the presence and absence of XOs. Both 20 and 200 μg/mL XOs suppressed the splenocytes proliferation, where activated splenocytes proliferated to the number of 9603±871, and addition of 20 and 200 μg/mL XOs reduced the counts to 1253±1038 (n=4, p<0.0001) and 1570±1010 (n=4, p<0.0001).

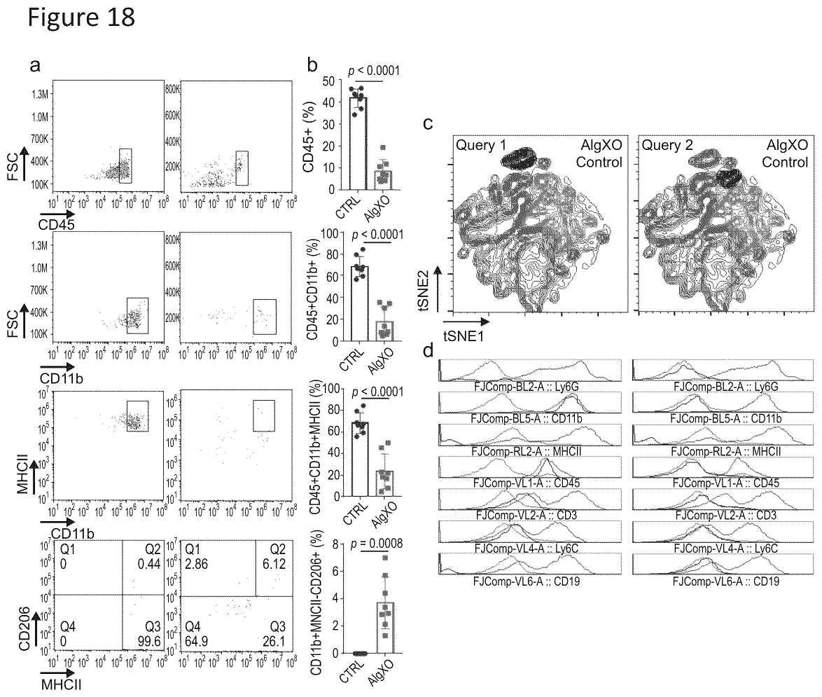

The cellular heterogeneity within splenocytes complicates the drawing of conclusion on the XOs cellular mechanism. To delineate more detailed cellular mechanisms underlying suppressive capabilities of XOs, we focused on the XOs effect on the activation of purified T cells. We thus repeated the T cells proliferation assay in purified T cells co-cultures, which gives insight onto the interactions between XOs and T lymphocytes. Purified CD3+ T cells were activated (similar to splenocyte activation procedure), and after 4 days the CD4+ counts for CD3/CD28 activated T cells was 5217±378. Addition of 20 and 200 μg/mL XOs reduced the counts to 3889±2081 (n=4, p=0.0031) and 4387±1397 (n=4, p=0.0057), respectively. Moreover, CD8+ counts for CD3/CD28 activated T cells was 2700±252 and addition of 20 and 200 μg/mL XOs reduced the counts to 1503±784 (n=4, p=0.0018) and 1766±628 (n=4, p=0.0002), respectively. Interestingly, XOs were more suppressive in the splenocytes co-cultures than purified T cells. These results suggest the remarkable involvement of non-T cells, including antigen-presenting cells (APCs), in the XOs suppressive mechanism in splenocyte co-cultures. This suggests that XOs, at least in part, target accessory cells such as APCs rather than T cells directly, which is in agreement with recent studies (Zhang, B. et al. Mesenchymal stromal cell exosome-enhanced regulatory Tcell production through an antigen-presenting cell-mediated pathway. Cytotherapy 20, 687-696 (2018); and Zhang, B. et al. Mesenchymal stem cells secrete immunologically active exosomes. Stem Cells Dev. 23, 1233-1244 (2014)). In a broader context, infused MSCs and their apoptotic products are suggested to be phagocytosed, leading to the generation of third-party phagocytes that ultimately mediate the observed immunomodulatory effects (de Witte, S. F. H. et al. Immunomodulation by therapeutic mesenchymal stromal cells (MSC) is triggered through phagocytosis of MSC by monocytic cells. Stem Cells 36, 602-615 (2018)). These observations imply that XOs first interface with APCs and phagocytes (Lankford, K. L. et al. Intravenously delivered mesenchymal stem cell-derived exosomes target M2-type macrophages in the injured spinal cord. PLoS ONE 13, e0190358 (2018); and Zhang, B. et al. Mesenchymal stromal cell exosome-enhanced regulatory Tcell production through an antigen-presenting cell-mediated pathway. Cytotherapy 20, 687-696 (2018)), which then facilitate the immunosuppression of T cells. These observations are in agreement with our earlier in vivo results, where CD3+ T lymphocytes were absent in the lavage collected from the AlgXO microcapsules microenvironment but were present in the lavage collected from the microenvironment of CTRL microcapsules (FIG. 18, d).

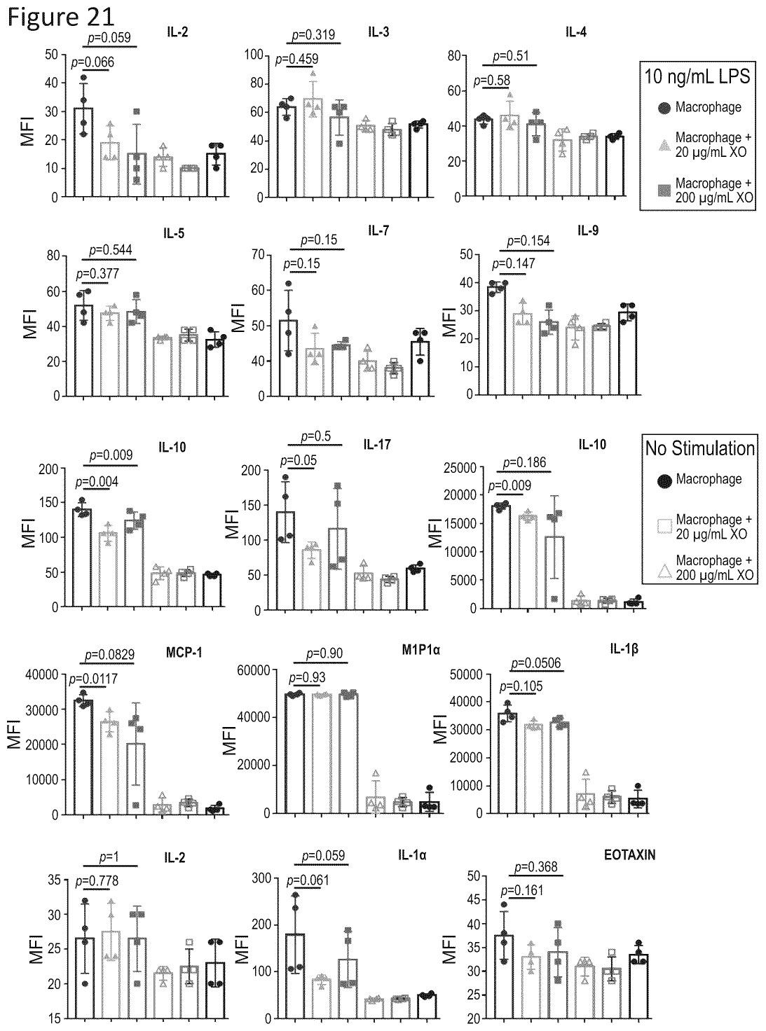

To functionally validate whether XOs possess immunomodulatory effects on APCs, and gain insight into XOs therapeutic mechanisms, we performed co-cultures of activated murine macrophages and XOs. Recently, the anti-inflammatory potentials of XOs derived from human-derived MSCs have been described in the LPS induced inflammation both in vitro cultures with murine macrophages and LPS injected mouse models (Pacienza, N. et al. In vitro macrophage assay predicts the in vivo anti-inflammatory potential of exosomes from human mesenchymal stromal cells. Mol. Ther. Methods Clin. Dev. 13, 67-76 (2019)). To gain insight into the possible mechanisms that XOs regulate macrophages activation, we isolated the supernatants from cocultures and measured the quantity of secreted cytokines. Among the panel of tested cytokines, we found that XOs significantly reduce the production of G-CSF, IFNγ, LIF, KC, MIP-2, RANTES, IL-6, LIX, and VEGF from LPS-stimulated macrophages (FIG. 5, e). LPS activates the NFκB pathway and all three MAPK pathways (ERK, JNK/SAPK, and p38a), leading to a wide range of cellular responses, including cell differentiation, survival or apoptosis, and inflammatory responses (Guha, M. & Mackman, N. LPS induction of gene expression in human monocytes. Cell. Signal. 13, 85-94 (2001)). Reduced cytokines and chemokines in macrophage culture are hallmarks of NFκB inflammatory pathway, suggesting that XOs likely possess anti-inflammatory properties through regulating this pathway (see Table 1). Inflammatory cytokines/chemokines that were not affected by XO addition include TNFα, IL-2, IL-17, and IL-la (FIG. 21). Interestingly, even the production of IL-10 was reduced by addition of XOs, demonstrating that in this specific experimental setting and timepoints, XOs have immunosuppressive roles.

### XOs Suppress Human T Lymphocytes and Regulate NFκB in Human Macrophages

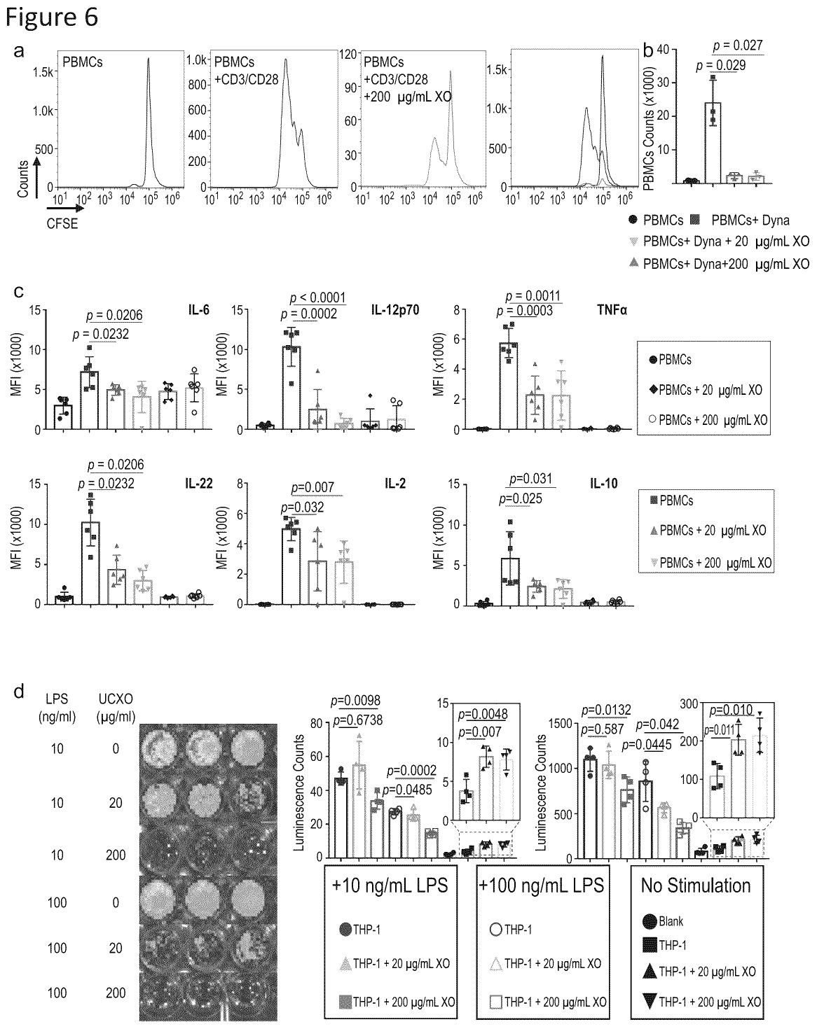

Our in vivo and in vitro assays thus far demonstrated the xenogeneic immunosuppressive capabilities of XOs. We further asked the replicability of such immunosuppressive potency of XOs on human-derived immunocytes, i.e., an allogeneic response. Since we observed a reduction of inflammatory response and an induction of tolerance in AlgXO transplanted mice in vivo, and reduction of murine T-cell proliferation and macrophage activation, we attempted to understand the immunomodulatory effects of XOs on human-derived immune cells in vitro. We examined XOs suppressive activity on T-cell proliferation ex vivo using carboxyfluorescein succinimidyl ester (CFSE)-labeled human peripheral blood mononuclear cells (PBMCs). PBMCs were activated with bead-bound anti-CD3/CD28 (1:1 ratio) and further cultured with or without XOs. Both 20 and 200 μg/mL XOs suppressed activation of PBMCs (FIG. 6, a). Quantitatively, addition of 20 and 200 μg/mL XOs reduced the count of activated T cells from 24002±6762 to 2342±910 (n=3; p=0.029) and to 2102±1121 (n=3; p=0.027), respectively (FIG. 6, b). These results are consistent with previous studies where the ability of MSC-derived exosomes to suppress T-cell activation and proliferation was reported (Riazifar, M. et al. Stem cell-derived exosomes as nanotherapeutics for autoimmune and neurodegenerative disorders. ACS Nano 13, 6670-6688 (2019); Hass, R., Kasper, C., Bohm, S. & Jacobs, R. Different populations and sources of human mesenchymal stem cells (MSC): a comparison of adult and neonatal tissue-derived MSC. Cell Commun. Signal 9, 12-12 (2011); and Bai, L. et al. Effects of mesenchymal stem cell-derived exosomes on experimental autoimmune uveitis. Sci. Rep. 7, 4323 (2017)). These results collectively suggest that XOs have potent suppressive effects on T cells activation, although the mechanisms behind such suppression remain to be fully understood.

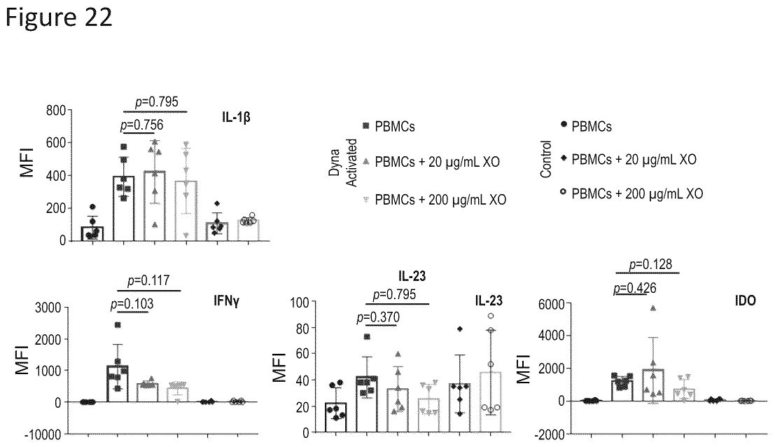

To gain a better understanding on the underlying cellular pathways, we performed Luminex assay to measure some cytokine profiles in the supernatant of PBMC co-cultures (FIG. 6, c and FIG. 22). We particularly examined cytokines that are related to macrophages and pro-inflammatory T lymphocyte subsets, such as Th1 and Th17 lymphocytes that play key roles in the FBR against biomaterials (Chung, L. et al. Interleukin 17 and senescent cells regulate the foreign body response to synthetic material implants in mice and humans. Sci. Transl. Med. 12, eaax3799 (2020); and Sommerfeld, S. D. et al. Interleukin-36γ-producing macrophages drive IL-17-mediated fibrosis. Science. Immunology 4, eaax4783 (2019)). In addition, recent reports have demonstrated the suppressive effects of XOs on Th1/Th17 cells polarization both in vitro and in vivo (Riazifar, M. et al. Stem cell-derived exosomes as nanotherapeutics for autoimmune and neurodegenerative disorders. ACS Nano 13, 6670-6688 (2019); Shigemoto-Kuroda, T. et al. MSC-derived extracellular vesicles attenuate immune responses in two autoimmune murine models: Type 1 diabetes and uveoretinitis. Stem Cell Rep. 8, 1214-1225 (2017); and Bai, L. et al. Effects of mesenchymal stem cell-derived exosomes on experimental autoimmune uveitis. Sci. Rep. 7, 4323 (2017)). To mechanistically probe the XOs effect in inhibiting the induction of T cell to Th1/Th17 subtypes, we measured several key representative Th1 and Th17 cytokines. In the presence of XOs, the levels of several pro-inflammatory Th1 and Th17 cytokines including IL-12p70 (Th1), TNFα (Th1), IL-6 (Th17), and IL-22 (Th17) were significantly reduced (FIG. 6, c). IFNγ (Th1) demonstrated a trend of decrease though not significant (FIG. 22). Interestingly, XOs significantly reduce the production of IL-2, which is a key cytokine to stimulate the growth, proliferation, and differentiation of T lymphocytes.

Cytokines are generally recognized as “signal 3”, which polarize helper T cells to Th1 (e.g., by IL-12 exposure) or Th17 (by IL-6 and IL-23) subsets. In addition, cytokines play a fundamental role in clonal expansion and persistence of antigen-reactive T lymphocytes and their effector activity. For instance, IFN-γ, IL-12, and IL-23 bind onto their receptors expressed on naive CD4+ T cells and drive the differentiation of Th1 cells through the activation of signal transducer and activator of transcription 1 (STAT1), STAT4, and T box transcription factor (T-bet) (Luckheeram, R. V., Zhou, R., Verma, A. D. & Xia, B. CD4+ T cells: differentiation and functions. J. Immun. Res. 2012, on the internet at: doi.org/10.1155/2012/925135 (2012); and Acharya, S. et al. Amelioration of Experimental autoimmune encephalomyelitis and DSS induced colitis by NTG-A-009 through the inhibition of Th1 and Th17 cells differentiation. Sci. Rep. 8, 7799 (2018)). Moreover, TNF-TNFR pairs control T-cell responses in two ways. First, they provide proliferative and survival signals either directly to the T cells or to the cognate APCs, regulating the frequency of effector and/or memory CD4+ or CD8+ T cells that can be differentiated from naive T cells in response to antigen stimulation. Second, they control T-cell function directly by promoting the production of cytokines such as IL-4 and IFNγ, or indirectly through stimulating the production of proinflammatory cytokines, such as IL-1 and IL-12, by professional or non-professional APCs (Croft, M. The role of TNF superfamily members in T-cell function and diseases. Nat. Rev. Immunol. 9, 271-285 (2009)). Upregulated IL-6 binds onto its receptor and activates retinoid-related orphan receptor γ T (RORγt) and STAT3, driving Th17 cell differentiation and function (Acharya, S. et al. Amelioration of Experimental autoimmune encephalomyelitis and DSS induced colitis by NTG-A-009 through the inhibition of Th1 and Th17 cells differentiation. Sci. Rep. 8, 7799 (2018); and Korn, T. et al. IL-6 controls Th17 immunity in vivo by inhibiting the conversion of conventional T cells into Foxp3 regulatory T cells. Proc. Natl Acad. Sci. USA 105, 18460 (2008)). Pathogenic Th17 cells are then polarized as a result of IL-23 and TGFβ3 stimulation (Lee, Y. et al. Induction and molecular signature of pathogenic TH17 cells. Nat. Immunol. 13, 991 (2012)).

We and others have observed that MSCs induced Treg expansion in a trans-well system only in the presence of splenocytes or peripheral blood monocytes, but not with purified CD4+ T cells (Riazifar, M. et al. Stem cell-derived exosomes as nanotherapeutics for autoimmune and neurodegenerative disorders. ACS Nano 13, 6670-6688 (2019); Tasso, R. et al. Development of sarcomas in mice implanted with mesenchymal stem cells seeded onto bioscaffolds. Carcinogenesis 30, 150-157 (2008); Tasso, R. et al. Mesenchymal stem cells induce functionally active T-regulatory lymphocytes in a paracrine fashion and ameliorate experimental autoimmune uveitis. Investigative Ophthalmol. Vis. Sci. 53, 786-793 (2012) and English, K. et al. Cell contact, prostaglandin E2 and transforming growth factor beta 1 play non-redundant roles in human mesenchymal stem cell induction of CD4+CD25Highforkhead box P3+ regulatory T cells. Clin. Exp. Immunol. 156, 149-160 (2009)). Exosome-treated monocytic THP-1 (but not MyD88-deficient THP-1) cells polarized activated CD4+ T cells to CD4+CD25+FoxP3+ Tregs at a ratio of one exosome-treated THP-1 cell to 1000 CD4+ T cells (Zhang, B. et al. Mesenchymal stem cells secrete immunologically active exosomes. Stem Cells Dev. 23, 1233-1244 (2013)). It is likely that XOs (as well as MSCs) play their immunosuppressive roles through interacting with myeloid lineage, and indeed, adoptive transfer of macrophages or monocytes, treated with MSC-EVs in vitro can protect the lung from injury (Mansouri, N. et al. Mesenchymal stromal cell exosomes prevent and revert experimental pulmonary fibrosis through modulation of monocyte phenotypes. JCI Insight 4, e128060 (2019)). We next sought to gain insight into the mechanisms by which XOs suppress macrophage activation by employing macrophages that produce luciferase in response to NFκB activation. FIG. 6, d shows the representative images of the luciferase activity (NFκB activity) as a result of 10 and 100 ng/mL LPS stimulation in the presence and absence of XOs. Luminescence count from IVIS imaging was quantified using equivalent regions on interest, suggesting that addition of 200 μg/mL of XOs reduces NFκB activation of both 10 ng/mL LPS (p=0.044) and 100 ng/mL LPS (p=0.004) activated THP-1 macrophages. Interestingly, 20 μg/mL of XOs did not efficiently reduce the NFκB activation. Same conditions were replicated, and signals were acquired using a plate reader, demonstrating a similar trend in the potency of XOs to inhibit NFκB activation (FIG. 6, d). Addition of 200 μg/mL XOs to the culture, reduced the luminescence counts of 10 ng/ml LPS activated THP-1 cells from 1097±64 to 762±71 (n=4, p=0.0132). Addition of 200 μg/mL XOs to the culture, reduced the luminescence counts of 100 ng/ml LPS activated THP-1 cells from 857±112 to 336±32 (n=4, p=0.0042). Surprisingly, XOs influenced the NFκB activation of non-activated THP-1 cells. Addition of 20 μg/mL XOs upregulated the NFκB activity in THP-1 cells from 109±17 to 203±20 (n=4, p=0.0117). Furthermore, addition of 200 μg/mL XOs upregulated the NFκB activity in THP-1 cells from 109±17 to 215±23 (n=4, p=0.0105). These results suggest that XOs could upregulate or downregulate the NFκB activity in macrophages, which partly recapitulates their parental MSCs, as MSCs themselves have been shown to regulate NFκB (Capcha, J. M. C. et al. Wharton's jelly-derived mesenchymal stem cells attenuate sepsis-induced organ injury partially via cholinergic anti-inflammatory pathway activation. Am. J. Physiol.-Regulatory, Integr. Comp. Physiol. 318, R135-R147 (2019)). NFκB controls multiple aspects of innate and adaptive immunity, and plays a critical role in regulating the function, activation, and survival of innate immunocytes and inflammatory T cells (Liu, T., Zhang, L., Joo, D. & Sun, S.-C. NF-κB signaling in inflammation. Signal Transduct. Target. Ther. 2, 17023 (2017)). NFκB pathway has been reported in response to PDMS (Moore, L. B., Sawyer, A. J., Charokopos, A., Skokos, E. A. & Kyriakides, T. R. Loss of monocyte chemoattractant protein-1 alters macrophage polarization and reduces NFkappaB activation in the foreign body response. Acta Biomater. 11, 37-47 (2015)), poly(ethylene glycol) (Amer, L. D. et al. Inflammation via myeloid differentiation primary response gene 88 signaling mediates the fibrotic response to implantable synthetic poly (ethylene glycol) hydrogels. Acta Biomater. 100, 105-117 (2019)), and alginate (Yang, D. & Jones, K. S. Effect of alginate on innate immune activation of macrophages. J. Biomed. Mater. Res. Part A 90A, 411-418 (2009); and Mohammadi, M. et al. Controlled release of stem cell secretome attenuates inflammatory response against implanted biomaterials. Adv. Healthc. Mater. 9, e1901874 (2020)), and reduction in NFκB has been correlated with reduced fibrosis (Moore, L. B. & Kyriakides, T. R. in Immune Responses to Biosurfaces (eds Lambris, J. D., Ekdahl, K. N., Ricklin, D. & Nilsson, B.) (Springer International Publishing, 2015); and Moore, L. B., Sawyer, A. J., Charokopos, A., Skokos, E. A. & Kyriakides, T. R. Loss of monocyte chemoattractant protein-1 alters macrophage polarization and reduces NFkappaB activation in the foreign body response. Acta Biomater. 11, 37-47 (2015)).

### Discussion

FBR against implanted materials creates patient discomfort and a variety of health complications (Mohammadi, M. R., Luong, J. C., Kim, G. G., Lau, H. & Lakey, J. R. T. in Handbook of Tissue Engineering Scaffolds, Vol. 1 (eds Mozafari, M., Sefat, F. & Atala, A.) (Woodhead Publishing, 2019); Swanson, E. Analysis of US Food and Drug Administration breast implant postapproval studies finding an increased risk of diseases and cancer: why the conclusions are unreliable. Ann. Plast. Surg. 82, 253-254 (2019) and Headon, H., Kasem, A. & Mokbel, K. Capsular contracture after breast augmentation: an update for clinical practice. Arch. Plast. Surg. 42, 532-543 (2015)). Moreover, if the goal is cell transplantation inside a biomaterial, FBR causes a non-functional graft engulfed in a scarring tissue (Bochenek, M. A. et al. Alginate encapsulation as long-term immune protection of allogeneic pancreatic islet cells transplanted into the omental bursa of macaques. Nat. Biomed. Eng. 2, 810-821 (2018)). This is one of the major challenges in clinical translation of tissue engineering and prosthesis products, sensors, and functional cell transplantation. One example is the alginate microcapsules, which has been under research for around 40 years (Franklin Lim, F. & Sun, A. M. Microencapsulated islets as bioartificial endocrine pancreas. Science 210, 908-910 (1980)). Some efforts over the past decade have determined that the FBR could be adjusted by purity of alginate (Paredes-Juarez, G. A., de Haan, B. J., Faas, M. M. & de Vos, P. A technology platform to test the efficacy of purification of alginate. Materials 7, 2087-2103 (2014)), microcapsules size64, surface chemistry (Vegas, A. J. et al. Combinatorial hydrogel library enables identification of materials that mitigate the foreign body response in primates. Nat. Biotechnol. 34, 345 (2016) and Liu, Q. et al. Zwitterionically modified alginates mitigate cellular overgrowth for cell encapsulation. Nat. Commun. 10, 5262 (2019)), and alginate composition (Farah, S. et al. Long-term implant fibrosis prevention in rodents and nonhuman primates using crystallized drug formulations. Nat. Mater. 18, 892-904 (2019)). We recently showed that even ultrapure alginate activates murine macrophages to secrete pro-inflammatory cytokines, and conditioned media secreted from UC-MSCs suppress such stimulation, partly through interfering with NFκB pathway (Mohammadi, M. et al. Controlled release of stem cell secretome attenuates inflammatory response against implanted biomaterials. Adv. Healthc. Mater. 9, e1901874 (2020)). Such cytokine secretion is not exclusive to alginate stimulation. Even human macrophages cocultured with endotoxin-free chitosan or poly(lactic acid) have reported to secrete IL-8, MIP-1, MCP-1, and RANTES or IL-6, IL-8, and MCP-197. Two mechanisms have been described to explain the reasons for alginate-based inflammatory response. Some studies have suggested the presence of immunogens within alginate (such as lipopolysaccharide (LPS), lipoteichoic acid, and peptidoglycans) are the main inducers of inflammation (Paredes-Juarez, G. A., de Haan, B. J., Faas, M. M. & de Vos, P. The role of pathogen-associated molecular patterns in inflammatory responses against alginate-based microcapsules. J. Control. Release 172, 983-992 (2013); and Paredes-Juarez, G. A., de Haan, B. J., Faas, M. M. & de Vos, P. A technology platform to test the efficacy of purification of alginate. Materials 7, 2087-2103 (2014)). Others reported that such contaminations were undetectable in their alginate (Doloff, J. C. et al. Colony stimulating factor-1 receptor is a central component of the foreign body response to biomaterial implants in rodents and nonhuman primates. Nat. Mater. 16, 671 (2017); and Vegas, A. J. et al. Combinatorial hydrogel library enables identification of materials that mitigate the foreign body response in primates. Nat. Biotechnol. 34, 345 (2016)), and linked the inflammatory response to the inherent properties of alginate. Alginate is a natural acidic polysaccharide extracted from marine brown seaweeds (Fang, W. et al. Identification and activation of TLR4-mediated signalling pathways by alginate-derived guluronate oligosaccharide in RAW264.7 macrophages. Sci. Rep. 7, 1663 (2017); and Bi, D. et al. Alginate enhances Toll-like receptor 4-mediated phagocytosis by murine RAW264.7 macrophages. Int. J. Biol. Macromol. 105, 1446-1454 (2017)). It is composed of different blocks of β-(1, 4)-Dmannuronate (M) and its C5 epimer α-(1, 4)-L-guluronate (G), and Guluronate oligosaccharide derived from alginate has been reported to readily activate macrophages partly through Toll-like receptor 4 (TLR4) signaling pathway (Fang, W. et al. Identification and activation of TLR4-mediated signalling pathways by alginate-derived guluronate oligosaccharide in RAW264.7 macrophages. Sci. Rep. 7, 1663 (2017); and Bi, D. et al. Alginate enhances Toll-like receptor 4-mediated phagocytosis by murine RAW264.7 macrophages. Int. J. Biol. Macromol. 105, 1446-1454 (2017)).

To this end, it can be claimed that an alginate formulation that lacks the inflammatory response could enhance its performance (e.g., functionality of the cell transplants) in immunocompetent rodents. Here we developed a hybrid platform of alginate that could release umbilical cord-derived MSC exosomes in a controlled manner. This platform reduces the inflammatory response against the xenotransplants, leading to >170 days glycemic control in the immunocompetent mouse model of T1D. Even single injection of XOs at the transplantation time delayed the graft rejection for ˜40 days on average. Resolving the inflammatory response to transplants have been demonstrated to extend the functional islet transplantation up to a year (Vegas, A. J. et al. Long-term glycemic control using polymer-encapsulated human stem cell-derived beta cells in immune-competent mice. Nat. Med. 22, 306-311 (2016); Farah, S. et al. Long-term implant fibrosis prevention in rodents and nonhuman primates using crystallized drug formulations. Nat. Mater. 18, 892-904 (2019); and Alagpulinsa, D. A. et al. Alginate-microencapsulation of human stem cell-derived (3 cells with CXCL12 prolongs their survival and function in immunocompetent mice without systemic immunosuppression. Am. J. Transplant. 19, 1930-1940 (2019)). The longevity and mechanism of local immunosuppression have been found to be among the key factors in determining the durability of transplanted islets.

To better understand the therapeutic mechanism of XOs on cellular level, we found that XOs immunosuppressive activity is more pronounce in the heterogenous population of splenocytes compared to when only CD3+ T cells present in the co-cultures (FIG. 5). While the effect of XOs on activated T cells is still debated (Zhang, B. et al. Mesenchymal stem cells secrete immunologically active exosomes. Stem Cells Dev. 23, 1233-1244 (2013) and Xie, M. et al. Immunoregulatory effects of stem cell-derived extracellular vesicles on immune cells. Front. Immunol. 11, 13-13 (2020)), the present study suggests that XOs are likely to play their immunosuppressive roles through interacting with myeloid lineage. This type of suppression has been widely reported to be due to induction of Tregs (Riazifar, M. et al. Stem cell-derived exosomes as nanotherapeutics for autoimmune and neurodegenerative disorders. ACS Nano 13, 6670-6688 (2019); Zhang, B. et al. Mesenchymal stromal cell exosome-enhanced regulatory Tcell production through an antigen-presenting cell-mediated pathway. Cytotherapy 20, 687-696 (2018); and Zhang, B. et al. Mesenchymal stem cells secrete immunologically active exosomes. Stem Cells Dev. 23, 1233-1244 (2013)), cell cycle arrest (Lee, S. et al. Mesenchymal stem cell-derived exosomes suppress proliferation of T cells by inducing cell cycle arrest through p27kip1/Cdk2 signaling. Immunol. Lett. 225, 16-22 (2020)), and adenosinergic immunosuppresion (Kerkelä, E. et al. Adenosinergic immunosuppression by human mesenchymal stromal cells requires co-operation with T cells. Stem Cells 34, 781-790 (2016)). Interfering with multiple signaling pathways not only makes XOs an exciting therapeutical biologic, but it also suggests potential multi-factorial side effects that could arise from such characteristic. Detailed mechanistical studies in the future need to address the therapeutic vs. side effect aspects of MSC-derived XOs in addition to the questions around their batch-to-batch variations and challenges associated with their storage.

While here the application of AlgXO is focused on the islet transplantation, its core technology could be broadly applicable to other areas of cells transplantation and implants rejection due to immune response.

### Supplemental Materials

We recently performed similar characterization for bone marrow derived MSC derived exosomes (Riazifar M, et al. Stem Cell-Derived Exosomes as Nanotherapeutics for Autoimmune and Neurodegenerative Disorders. ACS Nano 13, 6670-6688 (2019)) and microvesicles (Mohammadi M R, et al. Isolation and characterization of microvesicles from mesenchymal stem cells. Methods, (2019)), and found that Calnexin marker can be used as one of the markers to distinguish between exosomes and microvesicles as well as XOs purity. Comparing the Western blotting results from MSC derived MVs and MSC derived exosomes1 suggest that calnexin and CD81 may potentially be used to distinguish between exosomes and MVs. XOs were visualized and quantified using NTA analysis, where 1.7×10¹²±7.6×10¹¹ XOs/mL spherical particles with average diameter 105±48 nm were isolated from ˜150 to 190 million cultured MSCs in 100% confluency (FIG. 7, c). In developing methods to analyze XOs, we particularly sought to measure the expression of TGFβ-1 and PD-L1, as its expression on cancer cells XOs has been suggested to play a critical role in the immune evasion of tumor microenvironment (Daassi D, Mahoney K M, Freeman G J. The importance of exosomal PDL1 in tumour immune evasion. Nature Reviews Immunology, (2020); Chen G, et al. Exosomal PD-L1 contributes to immunosuppression and is associated with anti-PD-1 response. Nature 560, 382-386 (2018); and Haderk F, et al. Tumor-derived exosomes modulate PD-L1 expression in monocytes. Science Immunology 2, eaah5509 (2017)).

### Islet Dose Study

Clinical trials for islet transplantation have shown that allogeneic or xenogeneic source of islets affect the clinical efficacy and have resulted in conflicting results. More specifically, although xenotransplantation in non-immunosuppressed diabetic patients partially reduced hypoglycemic events, higher doses of xenogeneic islets were less effective (Matsumoto S, et al. Clinical Porcine Islet Xenotransplantation Under Comprehensive Regulation. Transplantation Proceedings 46, 1992-1995 (2014); and Ekser B, Bottino R, Cooper D K C. Clinical Islet Xenotransplantation: A Step Forward. EBioMedicine 12, 22-23 (2016)). Results from this trial showed that the transplantation of 5000 IEQ/kg xeno-islets was associated with superior glycemic control and graft function compared to higher doses of xeno-islets (i.e., 15,000 or 20,000 IEQ/kg). Interestingly, a recent auto-transplantation clinical trial demonstrated a strong dose-response relationship between the islet dose and graft function (Chinnakotla S, et al. Factors Predicting Outcomes After a Total Pancreatectomy and Islet Autotransplantation Lessons Learned From Over 500 Cases. Annals of Surgery 262, 610-622 (2015)). This trial suggested that the islet graft failure was 25-fold more likely in patients transplanted with low dose (<2000 IEQ/kg) islets versus higher doses (>5000 IEQ/kg or more) (Chinnakotla S, et al. Factors Predicting Outcomes After a Total Pancreatectomy and Islet Autotransplantation Lessons Learned From Over 500 Cases. Annals of Surgery 262, 610-622 (2015)). We thus sought to understand whether such observations could be replicated in our pre-clinical diabetic mice model, and we found that the xeno-islet dose is a critical determinant of the transplant therapeutic efficacy. We used a low dose (500 IEQ) and a high dose (5000 IEQ) islets transplanted within AlgXO and CTRL microcapsules. Islets (5000 IEQ) within AlgXO reversed hyperglycemia for about 80 days but failed to do so in longer periods. Surprisingly, 5000 IEQ islets within the CTRL microcapsules were not able to consistently reverse the hyperglycemia in STZ mice (FIG. 13, a). We further repeated the efficacy of AlgXO transplants in response to OGTT in the 5000 IEQ transplanted group and compared against non-diabetic control (FIG. 13, b). Polynomials with degree 5 were assigned to the OGTT curve of every individual mice (FIG. 13, c), and time to normoglycemia was calculated based on the value of 200 for the polynomial functions. FIG. 13, d demonstrates that the average time to reach normoglycemia after an OGTT for mice with AlgXO transplants was 112±32 minutes. This suggests a delay in glucose response of mice received AlgXO transplants versus non-diabetic mice (p=0.08). In addition, 6 out of 10 diabetic mice that received 5000 IEQ islets within CTRL microcapsules died within a day of transplantation, while this ratio was 1 out of 8 for AlgXO group (FIG. 13, e, p=0.0018). As a result, AlgXO microcapsules delayed the graft rejection and increased the normoglycemic duration in mice transplanted with high dose islets (FIG. 13, f); however, with less efficacy than medium dose of islets i.e., 1500 IEQ. We further tested lower dose of islets (500 IEQ), where neither the AlgXO nor the CTRL microcapsules were able to reverse hyperglycemia in the recipient mice (FIG. 13, g).

### Immune Microenvironment Around Subcutaneous Microcapsules

We found that total cell infiltration around microcapsules was significantly lower in AlgXO fibrotic tissues (p=0.011). Similar trends were observed for CD68 (p=0.037) and MHCII (p=0.015). In contrast, there was no association between CD206 expression (p=0.112). While these observations suggest the less immune-infiltrated milieu in AlgXO fibrotic microenvironment, the T cell sub population (CD3+) and fibrosis marker (αSMA) were found to be expressed more in AlgXO fibrotic microenvironment. These mixed outcomes were against our initial hypothesis on the anti-inflammatory and/or anti-fibrotic response of AlgXO microcapsules in vivo. In particular, αSMA, which was highly expressed in the AlgXO microenvironment, is a marker for activated myofibroblasts that are responsible for downstream collagen deposition and fibrosis of implanted alginate microcapsules (Doloff J C, et al. Colony stimulating factor-1 receptor is a central component of the foreign body response to biomaterial implants in rodents and non-human primates. Nature Materials 16, 671 (2017)). However, αSMA is also a contractile protein expressed in pericytes as well as in the vascular smooth muscle cells that surround arteries and arterioles (Kornfield T E, Newman E A. Regulation of Blood Flow in the Retinal Trilaminar Vascular Network. The Journal of Neuroscience 34, 11504 (2014)). In the histological observations, the αSMA cells were found to have a round structure consistent with blood vessel structure (FIG. 3, e). Next, we quantified the blood vessel formation and found that there is more blood vessel within the subcutaneous area (and around microcapsules) of AlgXO 2-weeks explants (FIG. 16, a). We further isolated cells from fibrotic tissues and analyzed their subpopulation using flow cytometry. There was significantly higher CD45+ cells (n=4; p<0.0001) collected from AlgXO fibrotic tissues (33.1%±8.0%) compared to control (83.0%±12.8%) (FIG. 16, b). Tissue sections were further analyzed for αSMA showing vasculature presence in the AlgXO fibrotic microenvironment, demonstrating a vascular-shaped microstructure (FIGS. 16, c and d). These results in their totality suggest the presence of blood vasculature and less inflammatory milieu around AlgXO fibrotic tissues.

We pursued the experiments to further investigate the anti-inflammatory properties of AlgXO microcapsules. Immune infiltration could be characterized with cell types present in the lavage around the inflamed area, particularly for biomaterials-based inflammation (Vegas A J, et al. Combinatorial hydrogel library enables identification of materials that mitigate the foreign body response in primates. Nature Biotechnology 34, 345 (2016); and Vegas A J, et al. Long-term glycemic control using polymer-encapsulated human stem cell-derived beta cells in immune-competent mice. Nature Medicine 22, 306-311 (2016)). Live cells within the subcutaneous lavage were first analyzed for common lymphocyte marker (CD45). FIGS. 18, a and b show the percentage of CD45+ cells in lavage of CTRL implanted mice were 41.6%±4.2% and in the AlgXO implanted were 8.5%±5.2% (n=4, p<0.0001). Sub-gating on CD45+ cells, the percentage of CD11b+ cells decreased from 68.4%±9.4% for CTRL to 17.6%±13.4% for AlgXO microcapsules (p<0.0001). Around 68.2%±9.5% of CD11b+ cells are also expressing MHCII for CTRL, while this percentage is 23.5%±16.3% for AlgXO microcapsules (p<0.0001). Interestingly, there was no detectable CD45+CD11b+MHCII-CD206+(M2-like macrophages (Vlahos A E, Cober N, Sefton M V. Modular tissue engineering for the vascularization of subcutaneously transplanted pancreatic islets. Proceedings of the National Academy of Sciences 114, 9337-9342 (2017))) for CTRL, while these macrophages 3.7%±1.9% for lavage retrieved from the surrounding environment of AlgXO microcapsules (p<0.0001).

To gain a more holistic information on the lavage immune-profile, we compared the lavage components of both microcapsules at the cellular level through tSNE representation. FIGS. 18, c and d show tSNE plots and two sub-populations that were analyzed for immune markers. Query 1 (gated on specific sub population present in CTRL but not in AlgXO) was CD45+CD11b+CD3−CD19−MHCII−Ly6C−Ly6G−, which is likely to be non-activated dendritic cells (Hey Y-Y, Tan J K H, O'Neill H C. Redefining Myeloid Cell Subsets in Murine Spleen. Front Immunol 6, 652 (2016)). Query 2 (gated on specific sub population present in AlgXO but not in CTRL) showed the subpopulation of cells with CD45−CD11b−CD3−CD19−MHCII−Ly6C−Ly6G− markers, which are likely to be from neither myeloid nor lymphoid origin. These results in their totality supports the reduced-inflammatory response against AlgXO implants, while non-inflammatory tissues were formed around AlgXO. It should be noted that transplantation of 1500 IEQ rat islets within AlgXO or CTRL failed to regulate the dysglycemia when transplanted subcutaneously (FIG. 19). Interestingly, while 1500 IEQ islets regulated mice hyperglycemia when transplanted intraperitoneally, subcutaneous transplantation failed to do so. A combination of stronger fibrotic response, lack of movability, and more hypoxic environment in the subcutaneous area are likely among the reasons for such a difference.

### Simulation of Release Model for Nanoparticle

To better understand the spatiotemporal profiles for the controlled release of XOs from AlgXO, we simulated such release using a MATLAB code. Simulations were run for homogenous spatially distributed XOs within AlgXO with diameter of 300 μm. Due to the 50-150 nm size distribution of XOs, we run the simulation with 50, 100, and 150 nm nanoparticle size. To further validate and characterize the release profiles other sizes (i.e., 10, 200, and 500 nm) were also tested in our simulation model. The 2.5% (weight/volume) alginate was used in our experimental studies. Thus, we used the same percentage for the porosity of alginate calculations (Equation 1).

### Modeling Assumptions

1. Microcapsule Size and Porosity

Simulations were run for uniformly sized and spatially distributed microcapsules and diameter was assumed to be 300 μm. Value of 2% (weight/volume) is evaluated as the default value for all simulations. Porosity of alginate solid is calculated by equation 1.

$$porosity = \frac{ρ1 - ρ2}{ρ1 - ρ3} \qquad ( 1 )$$

p1 is particle density, p2 is bulk density, and p3 is fluid density. For alginate solid, particle density is 1.6 g/ml, fluid density is density of water, which is 1 g/ml. Bulk density will be based on the concentration of alginate, which is shown in equation 2.

$$ρ2 = \frac{100 + {C}_{atg}}{100} \qquad ( 2 )$$

2. Surrounding Media

Assuming that capsules are implanted and surrounded by physiological fluid, surrounding viscosity was chosen to be 3.5*10⁻³ Pa*s.

3. Temperature

Assuming the capsule are implanted, temperature of microcapsule and surrounding environment should be close to body temperature, which is 37 degree.

4. XOs Concentration

XOs are homogeneously mixed inside the microcapsules, with an initial concentration of 10 particles/μm³.

5. XOs Size

XOs are generally recognized to be between 30-150 nm. Thus, we set up the particle diameter as 10 nm, 50 nm, 100 nm, 200 nm, and 500 nm to see the different result as particle size changes. Particles larger than 450 nm cannot diffuse out from the capsule (Fultz M J, Barber S A, Dieffenbach C W, Vogel S N. Induction of IFN-γ in macrophages by lipopolysaccharide. International Immunology 5, 1383-1392 (1993)).

6. Diffusion Model

To simplify our modeling, we just assumed that XOs diffuse out of microcapsules based on gradient density differences. We also assumed that a uniformed sphere symmetrically diffuses out in any direction. Thus, we established a 1-dimensional diffusion model for XOs.

### Mathematical Model Assumptions

1. 1-D Diffusion Equation

We are using heat equation to calculate the 1-dimensional diffusion of nanoparticles. Heat equation is a partial differential equation as shown in equation 3.

$$\begin{matrix}\partial C \\ \partial t\end{matrix} = D * \begin{matrix}{\partial }^{2} C \\ \partial {x}^{2}\end{matrix} \qquad ( 3 )$$

C is concentration gradient, t is time, and x is distance from center of the capsule. D is diffusion coefficient of XOs at certain position.

2. Diffusion Coefficient Outside the Microcapsule

To determine diffusion coefficient outside the capsule, we use Stroke-Einstein equation (equation 4).

$$D = \frac{R}{{N}_{A}} * \frac{T}{6 * π * η * r} \qquad ( 4 )$$

Where R is gas constant, NA is Avogadro constant, T is temperature in kelvin, η is viscosity of solution, and r is radius of XOs.

3. Effective Diffusion Coefficient Inside the Capsule

Effective diffusion coefficient inside a porous media is largely based on porosity and tortuosity of media. Generally, effective diffusion coefficient can be calculated based on equation 5.

$${D}_{eff} = D * \frac{porosity}{tortuosity} \qquad ( 5 )$$

For porous media, normally we have a relationship between porosity and tortuosity, which is shown in equation 6.

$$tortuosity = \text{?} \qquad ( 6 )$$

$$\text{?} \text{indicates text missing or illegible when filed}$$

Since the microcapsule diameter is ˜150 μm, the program will simulate concentration gradient from 0 to 500 μm. A 600 s run time was selected to visualize the concentration change inside the area. Diffusion of nanoparticle for 10 nm, 50 nm, 100 nm, 200 nm, and 500 nm is plotted (FIG. 4, h). As shown in the graph, nanoparticles with smaller diameter diffuse faster than which with larger diameter (FIG. 20). Particle with 500 nm diameter cannot diffuse out of the capsule. For particles with size of 10 nm, within 600 s, concentration of particles at center of the capsule will drop to 40% of initial concentration. For 50 nm nanoparticles, concentration at center of the capsule will drop to 80% of initial value. For 100 and 200 nm, there is no significant drop of concentration at center of the microcapsule. Concentration of nanoparticle outside the capsule also depends on particle size. For 10 nm nanoparticle, after 600 s, concentration of nanoparticle outside the capsule will larger than 15 particles/μm3. For 50 nm, 100 nm, and 200 nm, there is not enough nanoparticles 350 μm from center of the capsule (concentration <1 particle/μm3).

Through our high-throughput cytokine assay, we found that XOs significantly reduce the production of G-CSF, IFNγ, LIF, KC, MIP-2, RANTES, IL-6, LIX, and VEGEF from LPS stimulated macrophages (FIG. 5, e). These cytokines and chemokines are hallmarks of NFκB inflammatory pathway, suggesting that XOs likely possess anti-inflammatory properties through regulating this pathway. These cytokines could have complementary effects. For example, chemotactic signals include CXC chemokines such as CXCL1/KC, CXCL2/MIP-2, and CXCL5/LIX, and CXCL8/IL-8, which are potent chemoattractant for NGs and their increased production causes neutrophil granulocytes infiltration and extravasation (Amanzada A, Moriconi F, Mansuroglu T, Cameron S, Ramadori G, A Malik I. Induction of chemokines and cytokines before neutrophils and macrophage recruitment in different regions of rat liver after TAA administration. Laboratory Investigation 94, 235-247 (2014)). Table 1 summarizes the function of these cytokines and their relation to NFκB pathway.

TABLE 1
Macrophages Cytokines Influenced by XOs

| Chemokine/ | | |
|---|---|---|
| Cytokine | Function | Reference(s) |
| G-CSF/ | Regulates the survival, | (Lieschke G J, et al. Mice lacking |
| CSF3 | maturation, and proliferation | granulocyte colony-stimulating |
| | of neutrophil progenitors | factor have chronic neutropenia, |
| | Regulates the differentiation | granulocyte and macrophage progenitor |
| | of granulocyte lineages | cell deficiency, and impaired neutrophil |
| | Regulates neutrophils | mobilization. Blood 84, 1737-1746 |
| | mobilization from bone | (1994); and Chang S-F, Lin S-S, |
| | marrow to peripheral tissues | Yang H-C, Chou Y-Y, Gao J-I, Lu S-C. |
| | LPS-activated ERK2 functions | LPS-Induced G-CSF Expression in |
| | by remodeling local chromatin, | Macrophages Is Mediated by ERK2, |
| | interacting with C/EBPβ | but Not ERK1. PLOS ONE 10, e0129685 |
| | and synergizing its | (2015)) |
| | transactivation activity | |
| | to increase G-CSF expression | |
| IFNγ | The only known type II | (Fultz M J, Barber S A, Dieffenbach |
| | interferon | C W, Vogel S N. Induction of IFN-γ |
| | Upon binding to receptor, | in macrophages by lipopolysaccharide. |
| | JAK1 and JAK2 are | International Immunology 5, 1383-1392 |
| | activated and | (1993); and Platanias L C. Mechanisms |
| | phosphorylate STAT1 | of type-I- and type-II-interferon- |
| | Macrophages secrete upon | mediated signalling. Nature Reviews |
| | stimulation with LPS | Immunology 5, 375-386 (2005)) |
| LIF | LIF acts in an autocrine | (Nguyen HN, et al. Autocrine Loop |
| | manner via LIF receptor | Involving IL-6 Family Member LIF, |
| | to promote STAT4 activation. | LIF Receptor, and STAT4 Drives |
| | Activated STAT4 together | Sustained Fibroblast Production of |
| | with NF-kB/p65-p52 and | Inflammatory Mediators. Immunity 46, |
| | C/EBPb enhances IL-6 | 220-232 (2017)) |
| | transcription | |
| MIP-2/ | Important chemokine for | (Kim D-S, Ho Han J, Kwon H-J. NF-κB |
| CXCL2 | recruitment of neutrophils | and c-Jun-dependent regulation of |
| | NF-κB activation is | macrophage inflammatory protein-2 |
| | required for MIP-2 gene | gene expression in response to |
| | expression in the LPS- | lipopolysaccharide in RAW 264.7 |
| | signaling pathway A MIP-2 | cells. Molecular Immunology 388 40, |
| | promoter could be activated | 633-643 (2003)) |
| | by ectopical expression | |
| | of NF-κB p65 or c-Jun | |
| | transcription factors. | |
| RANTES | Secretes via LPS-induced | (Karlsen A, et al. Anthocyanins |
| | NF-kB activation in | Inhibit Nuclear Factor-κB |
| | monocytes through sterile α | Activation in Monocytes and Reduce |
| | and HEAT/Armadillo | Plasma Concentrations of Pro- |
| | motif-containing protein | Inflammatory Mediators in Healthy |
| | (SARM)toll/IL-1R domain- | Adults. The Journal of Nutrition 137, |
| | containing adaptor. | 1951-1954 (2007); and Gürtler C, et |
| | SARM is critical for | al. SARM regulates CCL5 production |
| | the recruitment of | in macrophages by promoting the |
| | transcription factors | recruitment of transcription factors |
| | and of RNA polymerase II | and RNA polymerase II to the Ccl5 |
| | to the Ccl5 promoter | promoter. The Journal of Immunology |
| | | 192, 4821 (2014)) |
| LIX/ | Important chemokine in | (Lin M, Carlson E, Diaconu E, Pearlman E. |
| CXCL5 | Neutrophil trafficking | CXCL1/KC and CXCL5/LIX are selectively |
| | | produced by corneal fibroblasts and |
| | | mediate neutrophil infiltration to |
| | | the corneal stroma in LPS keratitis. |
| | | Journal of leukocyte biology 81, |
| | | 786-792 (2007).; Wang L-Y, Tu Y-F, |
| | | Lin Y-C, Huang C-C. CXCL5 signaling |
| | | is a shared pathway of neuroinflammation |
| | | and blood-brain barrier injury |
| | | contributing to white matter injury |
| | | in the immature brain. J Neuroinflammation |
| | | 13, 6-6 (2016); and Chandrasekar B, et al. |
| | | Chemokine-Cytokine Cross-talk THE ELR+ |
| | | CXC CHEMOKINE LIX (CXCL5) |
| | | AMPLIFIES A PROINFLAMMATORY |
| | | CYTOKINE RESPONSE VIA A |
| | | PHOSPHATIDYLINOSITOL 3-KINASE- |
| | | NF-κB PATHWAY. Journal of |
| | | Biological Chemistry 278, |
| | | 4675-4686 (2003) |
| KC/ | CXCL1 is regulated through | (Bhattacharyya S, Borthakur A, Dudeja P K, |
| CXCL1 | interactions of NF-κB | Tobacman J K. Lipopolysaccharide-induced |
| | with other transcriptional | activation of NF-κB non-canonical |
| | regulatory molecules such | pathway requires BCL10 serine 138 and |
| | as poly(ADP-ribose) | NIK phosphorylations. Exp Cell Res 316, |
| | polymerase-1 (PARP-1) | 3317-3327 (2010); and Amiri K I, Richmond A. |
| | and cAMP response element | Fine tuning the transcriptional regulation |
| | binding protein (CREB)- | of the CXCL1 chemokine. Prog Nucleic Acid |
| | binding protein | Res Mol Biol 74, 1-36 (2003)) |
| VEGF | Important protein for | (Kiriakidis S, Andreakos E, Monaco C, |
| | angiogenesis | Foxwell B, Feldmann M, Paleolog E. VEGF |
| | VEGF production in human | expression in human macrophages is |
| | macrophages is NF-κB | NF-κB-dependent: studies using |
| | dependent and could be | adenoviruses expressing the endogenous |
| | significantly reduced | NF-κB inhibitor IκBα and a |
| | using the NF-κB | kinase-defective form of the IκB |
| | inhibitor, IκBα | kinase 2. Journal of Cell Science 116, |
| | | 665 (2003)) |

### Methods

Isolation and Characterization of UC-MSCs and their XOs.

Healthy pregnant women at full-term gestation (>37 weeks), maternal age 18-40 years old, and who gave birth at UCI Medical Center were chosen to be used for umbilical cord collection under IRB exemption #2016-2791. Any known complicated pregnancies were excluded from the collection. Umbilical cord-derived mesenchymal stem cells (UC-MSCs) were isolated according to the previously published method with some modifications (Lu, L.-L. et al. Isolation and characterization of human umbilical cord mesenchymal stem cells with hematopoiesis-supportive function and other potentials. Haematologica 91, 1017-1026 (2006)). Briefly, UCs were washed with PBS under a sterile laminar flow cell culture hood and were cut longitudinally to remove blood vessels. Tissues were then cut into 2-3-mm3 segments and incubated with 0.09% collagenase Type II (Sigma) for 45 min at 37° C. in a humidified incubator with 5% CO₂. After digestion, tissues were passed through 100-μm mesh-sized filters. Cells were then centrifuged at 300×g and 4° C. for 20 min and resuspended in DMEM/F12 (Gibco) supplemented with 10% FBS, 1% penicillin/streptomycin, and 1% L-glutamine. Cells transferred to 175-cm2 flasks and incubated at 37° C. in a humidified atmosphere with 5% CO₂. Flasks were left undisturbed for 2-3 days, after which the medium was changed to remove non-adherent cells. Adherent cells were then characterized for surface markers to further confirm their MSC origin. FIG. 7, a shows that isolated cells have low expression of Stro-1, high expression CD90/Thy1, CD146/MCAM, CD105/Endoglin, CD166, CD44 while cells are negative for CD19, CD45, and CD106. Such expression profiles are consistent with previous reports (Mennan, C., Garcia, J., Roberts, S., Hulme, C. & Wright, K. A comprehensive characterisation of large-scale expanded human bone marrow and umbilical cord mesenchymal stem cells. Stem Cell Res. Ther. 10, 99 (2019); and Lv, F. J., Tuan, R. S., Cheung, K. M. & Leung, V. Y. Concise Review: The surface markers and identity of human mesenchymal. Stem Cells Stem Cells 32, 1408-1419 (2014)). UC-MSCs were further cultured in serum-free media for 2 days. Next, exosome isolation was performed as based on reported methods (Riazifar, M. et al. Stem cell-derived exosomes as nanotherapeutics for autoimmune and neurodegenerative disorders. ACS Nano 13, 6670-6688 (2019); and Mohammadi, M. R. et al. Isolation and characterization of microvesicles from mesenchymal stem cells. Methods 177, 50-57 (2019)). Briefly, conditioned media from cultures of MSC were centrifuged at 300×g for 10 min. Supernatant was collected and transferred to ultracentrifuge tubes (Polyallomer Quick-Seal centrifuge tubes 25×89 mm, Beckman Coulter). Samples were then centrifuged in a Beckman Coulter ultracentrifuge (Optima L-90 K or Optima XE-90 Ultracentrifuge, Beckman Coulter) for 20 min at 16,500×g (Type Ti 45, Beckman Coulter), to remove microvesicles. Supernatant was then carefully collected and centrifuged for 2.5 h with a Type 45 Ti rotor at 4° C. at 120,000×g. Exosome pellet was resuspended in PBS and washed 1× at 4° C. at 120,000×g. The pellet was then resuspended in PBS and stored at −80° C. XOs were characterized according to an established protocol by International Society of Extracellular Vesicles, where CD63, TSG101, GAPDH, Galectin-1, and Hsp70 were present while and endoplasmic reticulum marker, Calnexin, was absent (FIG. 7, b). Twenty microliters of XO were mixed with 1×RIPA (Cell Signaling Technologies, USA) buffer and sonicated for 5 min, three times, with vortexing in between. Protein contents were measured using a BCA protein assay kit (Thermo Scientific Pierce, Rockford, IL, USA). Then, 25 μL of BSA standard or 25 μL of sample were transferred to a 96-well plate, and 200 ml working reagent was added. The plate was incubated for 30 min at 37° C. and absorbance was analyzed with a SpectraMax 384 Plus spectrophotometer at 562 nm and the Soft-Max Pro software (Molecular Devices, 1311 Orleans Drive, Sunnyvale, CA, USA). Twenty micrograms of protein were then subjected to electrophoresis on a gradient precast polyacrylamide gel (Mini-PROTEAN®; Bio-Rad Laboratories, Hercules, CA, USA). Samples were then transferred onto a nitrocellulose membrane which was then blocked with 5% Blotting Grade Blocker Non-Fat Dry Milk (Bio-Rad Laboratories) in Tris-buffer saline supplemented with %0.1 Polysorbate 20 (TBST) at 4° C. overnight. Membrane was washed with TBST following by incubation with primary antibodies against Calnexin (clone H-70; Santa Cruz Biotechnology, Santa Cruz, CA, USA), Galectin-1/LGALS1 (D608T), Rabbit mAb (Cat #12936), CD63 Rabbit mAb (Cat #EXOAB-CD63A-1), GAPDH Rabbit mAb (Cat #ab181602), Hsp70 Rabbit mAb (Cat #EXOAB-Hsp70A-1), and TSG101 (clone 4A10; Abcam, Cambridge, UK) dissolved in 0.25% Blotting Grade Blocker Non-Fat Dry Milk in TBST overnight at 4° C. Next, membrane was washed with TBST for 10 min, in triplicate. Secondary antibodies ECL anti-rabbit IgG horseradish peroxidase-linked F(ab′)2 fragment (donkey, anti-rabbit) (GE Healthcare, Buckinghamshire, UK) were diluted in 0.25% Blotting Grade Blocker Non-Fat Dry Milk in TBST and incubated for 1.5 h. Membranes were analyzed with ECL Prime Western Blotting Detection (GE Healthcare) and a VersaDoc 4000 MP (Bio-Rad Laboratories). XOs were labeled with anti-CD63-modified magnetic beads (Exosome Isolation CD63, Lot OK527, Life Technologies AS, Oslo, Norway) overnight with gentle agitation. The beads were washed with 1% exosome-depleted FBS in PBS and then incubated with human IgG (Sigma-Aldrich) for 15 min at 4° C. Following another washing step, the beads were incubated with PE-TGFβ, PE/Cy7-PD-L1, and APC/Cy7−MHCII or Isotype Controls (Biolegend, San Diego, USA) for 40 minutes with gentle agitation at room temperature. After another washing step, the samples were analyzed using a FACSAria (BD Bioscience) and data were processed using FlowJo Software (Tri Star, Ashland, OR, USA). Flow cytometry analysis of TGFβ, PD-L1, and MHCII expression on XOs bound to anti-CD63-coated beads demonstrated minimal expression of TGFβ-1 and MHCII, and the absence of PD-L1 (FIG. 7, d).

### Microcapsules Preparation

UPLVG alginate (NovaMatrix®, Sandvika, Norway) was fabricated by dissolving 2.5% w/v in 0.9% sterile saline solution and mounted on an air-driven electrostatic microcapsule generator (Nisco Engineering Inc., Oslo, Norway). The alginate solution was added dropwise into a sterile filtered (0.22 μm) gelling solution composed of sterile 20 mM barium chloride and 25 mM HEPES solution to generate circular microcapsules of ˜350 microns in diameter. AlgXO microgels were prepared by addition of 7.05×10¹⁰±3.69×10¹⁰ XOs/mL, thawed at RT for 10 to 20 min. Microcapsules were then washed via centrifugation at 100×g and 4° C. for 5 min.

### Dissolving Microcapsules and XOs Collection.

We first optimized microcapsule dissolution using EDTA chelator. EDTA (Sigma-Aldrich) with concentration of 0.5M was dissolved in DI water to make a stock solution. Dilutions were performed to test chelator activity at concentrations of 5 and 10 mM from the stock solution. One milliliter of each chelation solution was added on AlgXO or CTRL microcapsules (n=1000 microcapsules/group) and were tested under phase-contrast imaging. Images were obtained using EVOS Imaging system microscope (20/40 PH 2×; ThermoFisher Scientific), and images were captured at 1-min intervals. To determine the dissociation, images were taken of images were analyzed using a microcapsule analysis program (Microcapsule Analysis Program. v5.0.2) in ImageJ. Images were then quantified by the number of microcapsules detected by the program as previously described (Rodriguez, S. et al. Characterization of chelator-mediated recovery of pancreatic islets from barium-stabilized alginate microcapsules. Xenotransplantation 27, e12554 (2019)). Curves were made based on the following percentage: % of dissolved microcapsules=Detected microcapsules at t>0 Detected microcapsules at t¼0. Based on the results, microcapsules were dissolved for 10 min in 10 mM EDTA solution (FIG. 9). To quantify the XOs encapsulated within AlgXO, dissolved solution was then subjected to ultracentrifugation for 2.5 h at 4° C. and 120,000×g. Exosome pellet was resuspended in PBS and stored at −80° C. until further analyses.

### Rat Islet Quality Control and Viability.

Four- to six-week-old male Sprague-Dawley rats (Envigo Harlan, Houston, TX) were used as islet donors. Islet isolation was performed using standard collagenase digestion and gradient purification. Common duct was clamped on the side of mesoduodenum. Ice-cold collagenase V solution (6-7 ml with concentration of 1 mg/ml in HMS+) was injected into common bile duct (CBD) using 23G needle. Pancreas was then removed from dorsal wall of the abdominal cavity and transferred into 50 ml conical tube on ice box. Pancreas in the conical tubes were kept at 37° C. water bath (with 30 rpm shaking) for 17 min, after which 20 ml cold HMS+ was added into the conical tube and were hand-shaken strongly. Islets were then isolated and purified using noncontinuous density gradient. From each isolated batch, islets were tested for their quality including DTZ, viability, and glucose-stimulated insulin release (GSIR) assay were conducted. Upon each batch of islet isolation and prior to implantation, we ran quality control tests to identify the suitability of islets viability and function for transplantation. Islet count and purity (per rat pancreas) was 947±137 IEQ as measured using DTZ staining (FIG. 10). Viability of each isolation batch was more than 90%, and on average, it was 93%±2%. To quantify the viability of islets, 100 IEQ islets (either encapsulated or naked) were stained with Calcein AM (CalAM, Invitrogen, Cat #C1430) for live cells and propidium iodide (PI, Invitrogen, Cat #P3566) for dead and dying cells for 30 min. Stained islets were analyzed using a microplate reader (Tecan Infinite F200; Tecan). The islet viability was calculated by the equation: (CalAM+ cells)/(CalAM+ cells+PI+ cells)×100. GSIR assay was conducted to assure the islet quality prior to implantation. From each isolation batch, three technical replicates of 100 IE islets per sample were incubated at 37° C. and 5% CO₂ for 1 h in each media in the corresponding order: low glucose (2.8 mmol/L; L1), high glucose (28 mmol/L; H), high glucose plus 3-isobutyl-1-methylxanthine (28 mmol/L+0.1 mmol/L IBMX; H+), and last back to low glucose (2.8 mmol/L; L2). Supernatant was collected and stored at −20° C. until analysis. Insulin concentration released during incubation was measured using a porcine insulin enzyme-linked immunosorbent assay (Mercodia, cat #10-1200-01). Absorbance was then measured using a microplate reader with 450-nm wavelength filter (Tecan Infinite F200 and Magellan V7) and presented as (μg/L). Stimulation index (SI) was calculated as the ratio of insulin concentration secreted in high glucose over the insulin concentration secreted in the first low-glucose incubation. Our islet quality control criteria were: SI units >2, Viabilities >90%, and purities >90% (DTZ).

### Scanning Electron Microscopy.

Both AlgXO and CTRL microcapsules were airdried in an sterile chamber prior to SEM analysis. Dried samples were placed on carbon-tapped imaging stubs. We used Philips XL-30 FEG SEM with EDS (Noran 6) system, which is a thermionic field emission SEM with a fully automatic gun configuration controlled by advanced computer technology (the magnification is up to ×800,000 with 2-nm resolution). The working distance was adjusted to be 10 mm at 0.5 kV voltage, and 10 pA as the beam current.

### Streptozotocin Injection in Mice

C57/BL6 mice were fasted overnight (at least 12 h) prior to Streptozotocin (STZ; Sigma CAS #: 18883-66-4) injection. STZ (180 mg/kg of mice body weight) was dissolved in 10 ml STZ buffer (0.1M Sodium Citrate buffer pH=4.5) before injection. The buffer was vortexed and kept on ice for about 15 min prior to i.p. administration. To assure the STZ induction, mice had to be hyperglycemic for at least a week, and defined as non-fasting blood glucose levels >350 mg/dl from the tail vein. To minimize the surgery-induced mortalities, mice blood glucose was adjusted prior to transplantation via insulin injection. All the blood glucose reads in this study are non-fasting.

### Islet Transplantation.

Animal surgeries and protocols were carried out in compliance with all relevant ethical regulations, as approved by the UCI Committee on Animal Care (IACUC). STZ-induced diabetic or non-diabetic immune-competent (male C57BL/6 mice; Jackson Laboratory) with 8- to 10-week-age were anesthetized with 2.5% isoflurane, and then their abdomens (or top-backs) shaved and sterilized using betadine and 70% ethanol. Injections using 1 mL pipet were used for microcapsules (either with or without islets) transplantation into a 0.5 cm incision that was made along the top-back for implantation. For intraperitoneal transplantation, a 0.5-1.0 cm incision was created along the abdomen midline and the peritoneal wall followed by exposure to blunt dissection. Microcapsules were loaded into sterile pipette tip for injection. Then the peritoneal wall was closed with sutures.

### Glucose Tolerance Testing

Mice were fasted 10-14 h prior to oral glucose tolerance testing (OGTT) measurements. Next, a fresh glucose solution was prepared by dissolving 30% glucose in DPBS (3 mg/kg of mice body weight). Prior to glucose administration, mice blood glucose was measure. Mice were anesthetized with 2% isoflurane inhalation, and the glucose solution was orally injected by oral gavage. Next, blood glucoses were measured through tail-vein snipping upon 10, 20, 30, 60, 90, 120, and 180 min after glucose injection. Blood samples obtained from the tail vein were measured for glucose levels using a glucometer (CONTOUR® NEXT glucometer, Ascensia Diabetes Care, Parsippany, NJ).

### Fibrotic Tissue Sectioning

Fibrotic tissues (containing microcapsules) were cut and fixed in 4% PFA at 4° C. overnight. Next, tissues were washed with PBS 3× and embedded in 2% agar (CAT #: A1296, Sigma, USA). Agar molds were then embedded in plastic with wax. The entire cassette was placed in 58° C. paraffin bath for 15 min. Tissues were then sectioned with 7-μm thickness using an RM2255 microtome (Leica) with Superfrost slides. Prior to staining, an ethanol gradient dehydration and paraffin embedding cycle were performed.

### Lavage and Fibrotic Tissue Flow Cytometry

Prior to removing the implants from the subcutaneous or intraperitoneal areas, small incision was created on the distant site from the explants. One milliliter of cold DPBS was injected back and forth for 3× with pipette around the fibrotic microcapsules and suspended cells were removed and washed with DPBS. In the case of cytokine analysis, the lavage collected from intraperitoneal cavity was immediately frozen at −80° C. freezer until being shipped on dry ice to Eve Technologies (Calgary, Canada), where cytokines were analyzed using Mouse Focused 32-Plex Discovery Assay (CAT #: 17619). To analyze the cell populations, isolated cells were stained with CD3 (1:500 dilution, Biolegend Cat #: 100203), CD11b (1:200 dilution, Biolegend Cat #: 101211), I-A/I-E (1:200 dilution, Biolegend Cat #: 107628) CD19 (1:200 dilution, Biolegend Cat #: 115507) and CD206 (1:200 dilution, Biolegend, Cat #: 141711) in 2% BSA and 1% heat-inactivated FBS. Similar panel was used for the cells isolated from fibrotic tissues around microcapsules with a slight difference. To isolate cells from fibrotic tissues, they were first minced into 2-5 mm pieces and then microcapsules were dissolved using 10 mM EDTA (see FIG. 9). Clustering of flow cytometry data was completed by concatenating all 3 biological replicates into one file, and clustering with the tSNE (t-distributed stochastic neighbor embedding) plugin for 1000 iterations, operating at theta=0.5. Data are displayed as user-gated populations graphed against their respective X and Y tSNE coordinates.

### Click-iT Plus TUNEL Assay.

To analyze the viability of transplanted islets in vivo, we explanted microcapsules 1 month after transplantation, and conducted the TUNEL assay according to the manufacturer protocol (CAT #: C10617, Invitrogen). Briefly, microcapsules were washed 3× with ice-cold PBS and fixed in 4% formalin for 24 h at 4° C. Samples were permeabilized using 1×RIPA buffer for 20 min and rinsed with ice-cold PBS. TdT reaction was performed following by the Click-iT Plus reaction. Finally, DAPI counterstaining was conducted by 1:2000 dilution for 15 min, and microcapsules were images using Olympus FV3000 Laser-Scanning Confocal Spectral Inverted Microscope (Olympus, USA). Total signal area was then quantified using imageJ analyses, and area percentages were compared for islets in both AlgXO vs CTRL microcapsules.

### Controlled Release Studies

After fabrication, 1000 AlgXO and CTRL microcapsules were plated in a 6-well plate at 37° C. in a humidified incubator with 5% CO₂. At indicated timepoints after co-incubation (FIG. 4, g), 1 mL of the culture supernatant was collected and 1 mL of sterile DPBS was replaced inside the well to keep the culture volume constant. Plates were sealed to minimize the loss of water due to evaporation. Isolated media was then measured for total protein concentration, and exosomal content using NTA.

### Nanoparticle Tracking Analysis

NTA was performed using the Nanosight NS3000system (Malvern Instruments, USA). XOs (either from ultracentrifugation process or controlled-release experiment) were suspended in PBS to contain ˜10⁷-10¹⁰ particles per ml, which fits within the detection limits of Nanosight NS3000. Exosomes were analyzed based on light scattering using an optical microscope aligned perpendicularly to the beam axis. A 60-s video was recorded and subsequently analyzed using NTA software.

### Captive Bubble Contact Angle

We used a custom-made captive bubble, modifying the regular contact angel equipment (MCA-3, Kyowa Interface Science). Thin AlgXO and CTRL hydrogels were formed within a capillary space between two glass slides. Hydrogels on top of the glass slides were then merged into water beaker, and camera was focused on the hydrogel. Small air bubbles were then shut on the surface of hydrogel, creating the aqueous-solid-gas phase on the hydrogels surface. Images captured from the bubbles and contact angles were measured using ImageJ software with contact angle plugin, using circular and/or elliptical fits wherever appropriate.

### Microcapsule Mechanical and Physical Properties

Mechanical properties of microcapsules were measured using a microscale tension-compression test system (MicroTester G2, CellScale, Ontario, Canada). The probe was constructed by attaching a 1 mm×1 mm platen to a 154 μm cantilever and mounted to the instrument. Microcapsules were transferred by pipette into the test chamber, which was pre-filled with water. Single microcapsules were isolated using the platen-cantilever set-up, oriented by the attached microscope on the MicroTester to be in focus. The force as a function of time was measured for compressive strains of 0-50% using a 200 s loading time, a 10 s hold time, and a 20 s release time. Force resolution was adjusted at 1 μN and spatial resolution at 1.5 μm. Measurements were recorded at 200-ms intervals. The force-displacement data was then converted into stress-strain, with the associated curve used to obtain a linear regression line from the stress-strain curve with <0.2 strain.

### H&E, Masson's Trichrome and Immunofluorescence Staining

Trichrome staining was used to visualize collagen fibrosis around capsules. CTRL microcapsules were retrieved from mice after 2 weeks and fixed overnight using 4% paraformaldehyde at 4° C., following by embedding in paraffin and sectioning. Xylene was used to deparaffinize sections prior to tissue staining. hematoxylin and eosin (H&E) staining was done following the standard procedure, and slides were mounted using Permount (Fisher Scientific) and 0.17-mm glass coverslips. Then, tissue samples were mounted on slides, and imaged under Nikon Ti-E fluorescent Microscope (Leica, USA).

Immunofluorescence imaging was performed to determine immune populations infiltrated around microcapsules. Microcapsules collected after 2 weeks of subcutaneous implantation were then blocked in agar and underwent paraffin embedding process then cut and mounted. Alcohol and xylene processing were performed to deparaffinized the samples then the spheres underwent heatmediated antigen retrieval in pressure cooker with citrate buffer solution. The microcapsules were then blocked for 1 h using a 1% bovine serum albumin (BSA) solution. Next, tissue slides containing microcapsules were incubated for 1 h in an immunostaining cocktail solution consisting of DAPI (500 nM), αSMA (1:500 dilution, Biolegend Cat #: MMS-4665), CD68 (1:200 dilution, Biolegend Lot #: B229996), CD3 (1:500 dilution, Biolegend Cat #: 100203), CD11b (1:200 dilution, Biolegend Cat #: 101211), I-A/I-E (1:200 dilution, Biolegend Cat #: 107628), and CD206 (1:200 dilution, Biolegend, Cat #: 141711) in 2% BSA. To stain the microcapsules collected from i.p. cavity, they were washed three times with a 0.1% Tween 20 dissolved in 5% BSA solution and maintained in a 50% glycerol solution. Spheres were then transferred to glass slides and imaged using an Olympus FV3000 Laser-Scanning Confocal Spectral Inverted Microscope (Olympus, USA) equipped with 5 and ×10 objectives. 405, 488, and 640 nm solid-state lasers were used, and the laser power was adjusted to be 1-1.5% in all channels.

Protein adsorption was also conducted via co-incubation of IgG fluorescent antibody (PE mouse IgG1κ isotype ctrl clone: MOPC-21, Biolegend, CAT #: 400111, 1:200) with AlgXO or CTRL microcapsules for 24 h on a shaking plate at 37° C. Microcapsules were then washed 2× with 5 mL PBS and transferred to glass slides and imaged using an Olympus FV3000 Laser-Scanning Confocal Spectral Inverted Microscope (Olympus, USA). The 488 nm solid-state laser was used, and the laser power was adjusted to be 1-1.5%.

### Human PBMCs Proliferation and Cytokine Assay

Peripheral blood mononuclear cells (PBMCs) were isolated from buffy coats from healthy and anonymous blood donors (UCI Institute for Clinical and Transitional Science) by density gradient centrifugation (Ficoll-Pague plus, GE Healthcare). For proliferation assay, 20 μg of XOs were incubated with 1×10⁵ CFSE [5(6)-carboxyfluoresceindiacetate Nsuccinimidyl ester] (Molecular Probes, Eugene, OR) labeled PBMCs. To activate Tcell proliferation, Dynabeads™ Human T-Activator CD3/CD28 for T-cell expansion and activation was used with 1:1 ratio of PBMCs:Dynabeads™. PBMCs proliferation was analyzed after 4 days using flow cytometry (FACSAria, BD) and data were analyzed using the FlowJo. For cytokine analysis, cells were cultured in RPMI 1640 with 10% heat-inactivated FBS, 1% penicillin/streptomycin, and 1% L glutamine. Cells transferred to 96 or 48 well plates and incubated at 37° C. in a humidified atmosphere with 5% CO₂. Dynabeads Human T-Activator CD3/CD28 for T-cell expansion and activation was used with 1:1 ratio of PBMCs:Dynabeads. The XOs were mixed with fresh culture media (with 20 and 200 μg/mL concentrations). DynaBeads were then added to isolated PBMCs in the presence and absence of XOs. Supernatants were collected and Luminex assay was used to analyze the secreted cytokines. Fifty microliters of PBMC culture supernatants were collected and either frozen at −80° C. or immediately analyzed using a human custom ProcartaPlex (11plex, ThermoFisher Scientific, Vienna, Austria) with Luminex 77. Results were then reported as mean fluorescence intensity (MFI).

### Splenocytes and T-Cells Proliferation Assay

Spleens from FVB/n mice were purchased from the Jackson laboratory male mice were dissected, filtered into a single-cell suspension using 70 μm sterile filter, and red blood cells were removed using Tris-acetic-acid-chloride (TAC). Splenocytes were washed once with PBS and resuspended at 15×10⁶/mL in staining buffer (0.01% BSA in PBS). Splenocytes were stained with proliferation dye eFluor™ 670 (ThermoFisher Scientific, CAT #: 65-0840-85) using 5 mM dye per 10M cells and incubated in a 37° C. water bath for 10 min. Finally, cells were washed and resuspended at 1 M/mL in RPMI 1640 w/HEPES+L-glutamine (Gibco, CAT #: 22400-105) complete medium containing 10% FBS (Atlanta Biologicals, CAT #: S11150), 1× non-essential amino acids (Gibco, CAT #: 11146-050), 100 U/mL penicillin-100 μg/mL streptomycin (Gibco, CAT #: 15140163), 1 mM sodium pyruvate (Gibco, CAT #:11360-070), and 55 μM β-mercaptoethanol (Gibco, CAT #:21985-023), eFluor™ 670-labeled Splenocytes were plated (50×10³/well) in a U-bottom 96-well plate (VWR, CAT #: 10062-902) and activated with plate-bound anti-Armenian hamster IgG (30 μg/mL, Jackson Immuno Research, CAT #127-005-099) with CD3 (0.5 μg/mL, Tonbo, CAT #: 70-0031) and CD28 (1 μg/mL, Tonbo, CAT #: 70-0281). XOs with 20 or 200 μg/mL concentration were added to the co-cultures after cell seedings. After 4 days of culture, cells were stained with Zombie Live/Dead Dye (BioLegend, CAT #: 423105) and live cells were analyzed for proliferation.

Similar procedure was conducted for T lymphocytes, where isolated splenocytes were subjected to EasySep™ Mouse T cell Isolation Kit (StemCell Technologies, CAT #: 19851) according to the manufacturer's instructions. After 4 days of cocultures, T cells were collected and blocked with anti-mouse CD16/32 (BioLegend, CAT #: 101302), stained with Zombie Live/Dead Dye and fluorescent-conjugated antibodies: CD4 (BioLegend, CAT #: 100512; clone RM4-5) and CD8 (BioLegend, CAT #: 100709; clone 53-6.7). Cells were processed using the BD LSR II or BD LSRFortessa™ X-20 flow cytometer and analyzed using FlowJo software v10.0.7 (Tree Star, Inc).

### Macrophage Activation Assay

RAW 264.7 cells were purchased from ATCC (CAT #TIB-71) and NFκB reporter THP-1_Lucia human cell lines were purchased from InvivoGen (CAT #: thpl-nfkb) employed for downstream experiments of this study. Passages 5-10 were cultured in RPMI 1640 supplemented with 10% of heat-inactivated FBS in the presence of 1% penicillin/streptomycin and 1% L-glutamine. Cells were then stimulated with 10 or 100 ng/mL of LPS (Invitrogen, CAT #: 50-112-2025). Stimulated and non-stimulated cells were then mixed with XOs with the mentioned concentrations in the results section. Control cells, LPS-stimulated cells in the presence and absence of XOs, and non-stimulated cells in the presence and absence of XOs (100,000 cells for each condition) were co-cultured for 10-14 h at 37° C. in a humidified incubator with 5% CO₂. Next, supernatant was collected for cytokine analyses. Supernatants were centrifuged at 2500×g and 4° C. for 5 min and stored at −80° C. Samples were then shipped on dry ice to Eve Technologies (Calgary, Canada), where cytokines were analyzed using Mouse Focused 32-Plex Discovery Assay (CAT #: 17619).

### IVIS Imaging

NFκB reporter THP-1 Lucia human cell lines were used to measure the NFκB activity. These cells are engineered THP-1 monocyte cell line by stable integration of an NFκB-inducible Luc reporter construct. The levels of NFκB-induced secreted luciferase in the cell culture supernatant are readily assessed with Quanti-Luc (CAT #: rep-qlc2). As a result, these cells could quantitatively measure NFκB activation. Cell were cultured in a phenol-free media and supernatants (as described in the in vitro co-culture section of the “Methods”) were collected. QUANT-Luc assay solution was added with a concentration of 1 mg/mL and incubated for 30 s. The resulted plate was then imaged in an IVIS imager (or VersaDoc 4000 MP). Exposure time was adjusted as 0.2 s, field of view 12.5, f number 16, and binning factor of 4 were selected as optimized acquisition settings.

### Animal Studies

All animal procedures were performed under approved University of California Irvine, Institutional Animal Care and Use Committee (Protocol #: AUP-17-241), in accordance with the guidelines of the National Institutes of Health.

What is claimed is:

1. A hybrid microcapsule comprising:
(a) a shell that comprises one or more biocompatible material,
(b) exosomes contained within the microcapsule, and
(c) one or more therapeutic cells encapsulated within the microcapsule, wherein the therapeutic cells are capable of releasing one or more therapeutic agent(s).

2. The hybrid microcapsule according to claim 1, wherein the one or more biocompatible material is a natural material selected from the group consisting of alginate, pectin, agarose, collagen and hyaluronic acid or a synthetic material selected from the group consisting of poly(ethylene glycol) (PEG), 2-hydroxyethyl methacrylate (HEMA) and poly(lactic-co-glycolic acid) (PLGA).

3. The hybrid microcapsule according to claim 2, wherein the one or more biocompatible material comprises an alginate or a derivative thereof.

4. The hybrid microcapsule according to claim 3, wherein the alginate or derivative thereof is a cross-linked ultrapure alginate.

5. The hybrid microcapsule according to claim 1, wherein the outer surface of the shell is hydrophilic and resistant to protein binding.

6. The hybrid microcapsule according to claim 1, wherein the exosomes are derived from mesenchymal stem cells (MSCs).

7. The hybrid microcapsule according to claim 6, wherein the mesenchymal stem cells are umbilical cord mesenchymal stem cells.

8. The hybrid microcapsule according to claim 7, wherein the umbilical cord mesenchymal stem cells are human umbilical cord mesenchymal stem cells.

9. The hybrid microcapsule according to claim 1, wherein the exosomes have a particle diameter of from 10-500 nm.

10. The hybrid microcapsule according to claim 9, wherein the exosomes have a particle diameter of from 20-200 nm.

11. The hybrid microcapsule according to claim 1, comprising 1×10⁵-1×10⁸ of the exosomes within the microcapsule.

12. The hybrid microcapsule according to claim 1, wherein the one or more therapeutic cells comprise pancreatic islets.

13. The hybrid microcapsule according to claim 12 wherein the microcapsule comprises 1-10 islet equivalent (IEQ) cells.

14. A method of making the hybrid microcapsule according to claim 1 comprising:
(a) isolating exosomes from mesenchymal stem cells (MSCs),
(b) obtaining therapeutic cells that are capable of releasing the one or more therapeutic agent(s), and
(c) incorporating the exosomes and the therapeutic cells into a microcapsule.

15. The method according to claim 14, wherein the microcapsule is an alginate microcapsule.

16. The method according to claim 14, wherein the MSCs are umbilical cord derived MSCs (UC-MSCs).

17. A method of treating a subject comprising administering the hybrid microcapsule according to claim 1 to the subject, wherein the therapeutic cells contained within the hybrid microcapsule release the therapeutic agent to the subject and wherein the hybrid microcapsule releases the exosomes to effectively attenuate an immune-based foreign body response (FBR) and enhance the viability of the encapsulated therapeutic cells.

18. The method according to claim 17, wherein the therapeutic cells are pancreatic islets and wherein the subject is treated for Type 1 diabetes.

19. A method of attenuating an immune response to a microcapsule in a subject comprising administering a microcapsule comprising exosomes contained within the microcapsule to the subject, wherein the exosomes are released from the microcapsule and wherein, upon release, the exosomes suppress a local immune microenvironment and effectively attenuate the immune response.

20. The method according to claim 19, wherein the immune response to the microcapsule is an immune-based foreign body response (FBR) to biomaterials in the microcapsule.
